(19) United States
(12) Patent Application Publication
(10) Pub. No.: US 2026/0327856 A1
(43) Pub. Date: Oct. 22, 2026

(54) VARIANTS OF ANKYRIN REPEAT DOMAINS

(71) Applicant: ATHEBIO AG, Zurich-Schlieren (CH)

(72) Inventors: Patrik FORRER, Zurich-Schlieren (CH); Rohan Sakariah EAPEN, Zurich-Schlieren (CH)

(21) Appl. No.: 19/099,653

(22) PCT Filed: Aug. 16, 2023
(86) PCT No.: PCT/EP2023/072510

§ 371 Date: Jan. 29, 2025

(30) Foreign Application Priority Data
Aug. 16, 2022 (WO) PCT/EP2022/072884
Feb. 13, 2023 (WO) PCT/EP2023/053418

(51) Int. Cl. C40B 30/04; A61K 38/00; C07K 14/47; C40B 40/10

(52) U.S. Cl. CPC C40B 30/04; C07K 14/47; C40B 40/10; A61K 38/00

(57) ABSTRACT

Described herein are proteins comprising an ankyrin repeat domain with a mutation in the N-terminal capping module, in particular a mutation at position 17 of the N-terminal capping module, as well as related products and methods.

### FIELD OF THE INVENTION

The present invention relates to variants of an ankyrin repeat domain and related products and methods. In particular, the present invention relates to an ankyrin repeat domain having a mutation at position 17 of the N-terminal capping module.

### BACKGROUND OF THE INVENTION

Different classes of specific binding proteins have evolved in nature, the most widely known class being immunoglobulins of vertebrates. Another class of specific binding proteins are repeat proteins. Similar to the role that immunoglobulins play in vertebrates, repeat proteins were found to be involved in the adaptive immune system of jawless fish. However, repeat proteins play a much wider role beyond this function and mediate protein-protein interactions across all phyla to fulfill diverse biological functions. In fact, they constitute the largest group of natural proteins mediating specific binding (e.g. reviewed in Forrer, P., et al., FEBS letters 539, 2-6, 2003). Repeat proteins bind their targets via the repeat domain, which is made up of a variable number of repeats that stack on each other through their conserved interfaces to create the compactly folded repeat domain. Specific target binding is then achieved through variable residues on the surface of the repeat domain (Forrer 2003, loc. cit, and WO 2002/020565).

Ankyrin repeat proteins are a well-studied class of repeat proteins (e.g. Binz, H. K., et al., Nat. Biotechnol. 22, 575-582, 2004 and Mosavi, L. K., et al., Protein Sci. 2004 June; 13 (6): 1435-48). The ankyrin repeat usually comprises 33 amino acid residues forming two antiparallel alpha-helices and a beta-turn. The folded ankyrin repeat domain comprising the stacked ankyrin repeats has a right-handed solenoid structure with a compact hydrophobic core and a large binding surface, which allows it to adapt to its respective binding partners. The terminal capping modules of the ankyrin repeat domain usually have a divergent sequence with polar residues to facilitate interaction with the solvent, thus capping the hydrophobic core. The basic architecture of the ankyrin repeat domain is shown in FIG. 1.

Plückthun and coworkers derived a consensus sequence motif from naturally occurring ankyrin repeats (e.g., Binz, H. K., et al., J. Mol. Biol., 332, 489-503, 2003 and WO 2002/020565). The derived ankyrin repeat consensus motif is 33-amino acid residues long and comprises fixed and variable positions (the latter also being referred to as randomized positions). The fixed positions correspond mainly to framework residues that are primarily responsible for the structural integrity of the ankyrin repeat domain, in particular, for the interpret stacking interactions. The variable positions correspond to surface-exposed residues that do not strongly contribute to the structural integrity of the ankyrin repeat domain but are potentially involved in target binding (though surface-exposed framework residues may also be involved in target binding).

Libraries of proteins, each having an ankyrin repeat domain with internal ankyrin repeats that were based on such ankyrin repeat consensus motif, were then created (Binz, 2004, loc. cit.). Certain variable positions of the consensus motif were randomized in each internal ankyrin repeat to allow for the binding to different targets, thereby creating the diversity of the library. In order to avoid aggregation of ankyrin repeat domains consisting only of internal ankyrin repeats, the internal ankyrin repeats were flanked by an N-terminal capping module and a C-terminal capping module to shield the hydrophobic core of the domain from the solvent (Forrer, 2003, loc. cit, and Binz, 2003, loc. cit.). These capping modules were based on the capping repeats from the guanine-adenine-binding protein (GA-binding protein), a naturally occurring ankyrin repeat protein (PDB: 1AWC_B).

Though the recombinant ankyrin repeat scaffold originally conceived by Pluckthun and colleagues was already stable, various mutations have been reported that yet further increase thermostability of such proteins. Interlandi et al. reported stabilizing mutations in the C-terminal capping module (Interlandi, G., et al., J Mol Biol. 2008 Jan. 18; 375 (3): 837-54). Similarly, stabilizing mutations in the N-terminal capping module have been reported (WO 2012/069655; WO 2022/038128; WO 2022/219185; PCT/EP2022/072884 and Schilling, J., et al., J Biol Chem. 2021 Nov. 15; 298(1):101403).

There remains a need to further improve the properties of proteins comprising an ankyrin repeat domain, such as the thermostability of the ankyrin repeat domain.

### SUMMARY OF THE INVENTION

The present invention is based on the discovery that specific mutations in the N-terminal capping module can significantly improve the properties of an ankyrin repeat domain. In particular, it has been found that the amino acid residue at position 17 of the N-terminal capping module is of key importance for the thermostability of the ankyrin repeat domain.

An amino acid residue selected from the group consisting of L, I and V at this position has been found to be particularly favorable for the thermostability of the ankyrin repeat domain. Furthermore, the effects of this mutation were transferable to ankyrin repeat domains with diverging sequences, demonstrating the general importance of this position in the N-terminal capping module for the thermostability of ankyrin repeat domains.

Accordingly, the present invention provides an ankyrin repeat domain that has an N-terminal capping module with a mutation at position 17, such as an amino acid residue selected from the group consisting of L, I and V, as well as a protein comprising such domain.

In further aspects, the present invention provides a protein library comprising such protein and a method of selection using such protein library.

The present invention also provides a pharmaceutical composition comprising the protein of the invention, a nucleic acid encoding it and a vector or cell comprising such nucleic acid.

In a further aspect, the present invention provides a method of preparing a protein of the invention comprising culturing a cell having a nucleic acid encoding the protein under conditions allowing expression thereof and then purifying the expressed protein.

In a further aspect, the present invention relates to the protein of the invention for use in a method of treatment.

Related products and methods are also provided, as will be apparent from the following detailed description.

### BRIEF DESCRIPTION OF THE FIGURES

FIG. 1: The basic architecture of an ankyrin repeat domain. One or more internal ankyrin repeats stack on each other (and the terminal capping modules) to form a hydrophobic core, which gets shielded on both ends from the solvent by terminal capping modules. The variable surface residues allow the ankyrin repeat domain to bind to different targets.

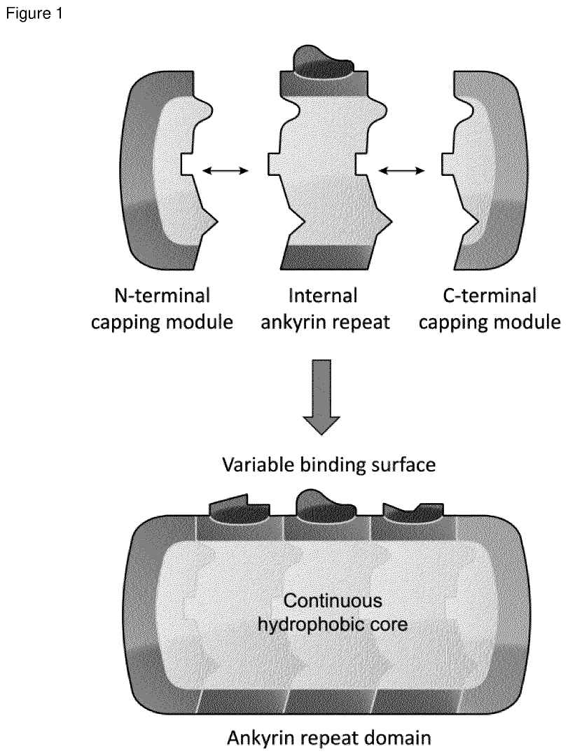

FIG. 2: The archetypal designed ankyrin repeat domain sequence of the N-terminal capping module as described by Binz et al. (Binz, 2003, loc. cit.), which corresponds to SEQ ID NO: 42. Position 17 of the N-terminal capping module is highlighted. The original amino acid at this position 17 is E.

FIG. 3: Exemplary sequence alignment of SEQ ID NO: 1, an N-terminal capping module sequence, and SEQ ID NO: 119 representing an entire ankyrin repeat domain. SEQ ID NO: 1 and SEQ ID NO: 119 have 23 out of 30 identical amino acid residues across the alignment window (i.e. 76% sequence identity).

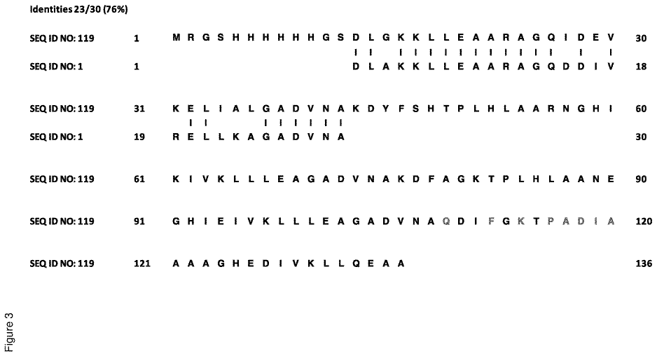

FIG. 4: Thermal stability of the ankyrin repeat domains P #104, P #105, P #106, P #107 and P #108, which have an identical sequence except for position 17 of the N-terminal capping module, which is occupied by E, I, V, L and T, respectively. Traces from thermal denaturation of P #104, P #105, P #106, P #107 and P #108 are shown. The Tm values for P #104, P #105, P #106, P #107 and P #108 were determined to be 72.2° C., 73.6° C., 73.2° C., 75.4° C. and 70.7° C., respectively, in PBS containing 4 M GdmCl. FF, fraction folded in %; T, temperature in ° C.

### DEFINITIONS

“A”, “an”, and “the” include plural reference unless the context clearly dictates otherwise. Thus, for example, reference to a protein comprising an ankyrin repeat domain refers to one or more such proteins.

The internal ankyrin repeat that is “adjacent” to the N-terminal capping module refers to the internal ankyrin repeat that is directly C-terminal of the N-terminal capping module forming an interface with the N-terminal capping module. Similarly, the internal ankyrin repeat that is “adjacent” to the C-terminal capping module refers to the internal ankyrin repeat that is directly N-terminal of the C-terminal capping module forming an interface with the C-terminal capping module.

The amino acid residues are referred to herein interchangeably by their full name, their three-letter code or their one-letter code. The “naturally occurring amino acid residues” refer to the twenty amino acid residues that are most commonly found in nature, i.e. A, R, N, D, C, E, Q, G, H, I, L, K, M, F, P, S, T, W, Y and V.

An “ankyrin repeat” refers to a short sequence of amino acid residues forming a structural motif. Ankyrin repeats occur in consecutive copies, are involved in protein-protein interactions and the core of the ankyrin repeat forms a helix-loop-helix structure (e.g., SMART accession number: SM00248).

The term “ankyrin repeat domain” refers to a protein domain comprising an N-terminal capping module, a C-terminal capping module and one or more ankyrin repeats in between (also referred to as “internal ankyrin repeats”). The folded ankyrin repeat domain has a right-handed solenoid structure with a large binding surface that is adaptable to specifically bind targets. The ankyrin repeat domain is generally very robust and can sustain a significant number of mutations, including substitutions, additions and deletions, without destroying its overall structure or function. The residues that contribute to the structural integrity of the ankyrin repeat domain, including the interpret interactions, are referred to as “framework residues”, whereas the residues that contribute to target binding, either through direct interaction with the target or by influencing residues that directly interact with the target, e.g., by stabilizing them, are referred to as “target interaction residues”. A single amino acid residue can be both—a framework and a target interaction residue—at the same time and framework residues and target interaction residues may be found not only in the internal ankyrin repeats, but also the N-terminal capping module and/or the C-terminal capping module.

The internal ankyrin repeats contribute to the structural stability of the ankyrin repeat domain through their stacking interactions with the neighboring repeats. An internal ankyrin repeat usually consists of 33 amino acid residues.

The capping modules have a hydrophobic inside surface that is suitable for interacting with the adjacent internal ankyrin repeat and a hydrophilic outside surface to shield the hydrophobic core from the solvent. In some embodiments, the N-terminal capping module and/or the C-terminal capping module are a N-terminal capping repeat and/or C-terminal capping repeat, respectively, which have a similar or the same fold as the adjacent internal ankyrin repeat(s) and/or sequence similarities to said adjacent internal ankyrin repeat(s).

The terms “binding”, “specific binding” or the like when used in reference to a target mean a binding interaction that is measurably different from a non-specific interaction, e.g., the interaction with a control molecule that is unrelated to the specific target. Control molecules that are commonly used to measure such non-specific interaction include bovine serum albumin, bovine casein and Escherichia coli (E. coli) maltose binding protein. In certain instances, the terms “binding”, “specific binding” or the like mean that only the target is bound and substantially no other molecule. Specific binding can be determined, for instance, by measuring the dissociation constant (Kd) for the target and/or by comparing the binding to the target with the binding to a control molecule. The Kd can be measured by various conventional techniques, such as isothermal titration calorimetry, radioligand binding assay, fluorescence resonance energy transfer, and surface plasmon resonance. The binding specificity is generally measured in standardized solutions, such as PBS. For instance, the Kd for the target in PBS is at least 10, at least 102, at least 10³ or at least 104 times lower than the corresponding Kd for a control molecule that is unrelated to the specific target.

The term “designed ankyrin repeat protein” or “DARPin” refers to a non-natural protein comprising an ankyrin repeat domain. In some embodiments, such a DARPin has a repeat sequence motif that was derived from natural ankyrin repeats, e.g. by consensus design (see, e.g., Forrer et al., 2004 Chem Bio Chem, 5, 2, 183-189 and Binz 2003, loc. cit).

The term “fraction of refolded ankyrin repeat domains after thermal denaturation” refers to the fraction of ankyrin repeat domains that refold into their native state after thermal denaturation.

The term “HEPES” refers to 4-(2-hydroxyethyl)-1-piperazineethanesulfonic acid. In some embodiments, a HEPES buffer contains 10 mM HEPES and 150 mM NaCl and has a pH of 7.4.

The term “library” as used in reference to a protein or nucleic acid library refers to a collection of proteins and nucleic acids, respectively.

The term “melting temperature” or “Tm” refers to the temperature at which 50% of the protein is unfolded in a certain buffer, e.g., PBS.

The term “PBS” refers to phosphate-buffered saline. In some embodiments, PBS contains 137 mM NaCl, 10 mM phosphate and 2.7 mM KCl and has a pH of 7.4.

The term “percent (%) sequence identity” with respect to a reference amino acid sequence specified herein is defined as the percentage of amino acid residues in a candidate amino acid sequence that is identical with the amino acid residues in the reference amino acid sequence, after aligning the sequences and introducing gaps, if necessary, to achieve the maximum percent sequence identity, and not considering any conservative substitutions as part of the sequence identity. In some embodiments, such alignment comprises no gaps. Unless specified otherwise, the comparison window is the entire length of the reference amino acid sequence. Alignment for purposes of determining percent amino acid sequence identity can be achieved in various ways that are within the skill in the art, for instance, using publicly available computer software such as BLAST, BLAST-2, ALIGN or GenePAST. In some embodiments, the GenePAST algorithm, formerly known as KERR algorithm (Dufresne G, et al. Nat Biotechnol. 2002 December; 20(12):1269-71), is used for alignment purposes. Those skilled in the art can determine appropriate parameters for measuring alignment, including any algorithms needed to achieve maximal alignment, in particular, over the full length of the reference amino acid sequence. Further examples of how to determine the percentage of sequence identity can be found in WO 2009/058564 A2, page 93, line 14 to page 102, line 5. When determining the sequence identity between a reference sequence and a candidate sequence, it is understood that if an “X” in a reference amino acid sequence, such as in SEQ ID NO: 61, is further defined as being selected from a certain group of amino acid residues, e.g. any naturally occurring amino acid residue, the “X” is counted as a match in a sequence alignment if the amino acid residue of the candidate sequence is identical to one of the amino acid residues defined for this position in the reference sequence.

The term “pharmaceutically acceptable carrier” refers to buffers, carriers, and other excipients suitable for use in contact with tissues of humans and/or animals without excessive toxicity, allergic response, irritation, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration.

The term “pharmaceutical composition” refers to a composition comprising at least one active agent and, generally, at least one pharmaceutically acceptable carrier. A pharmaceutical composition is generally formulated and administered to exert a pharmaceutically useful effect while minimizing undesirable side effects.

The “position(s)” of the N-terminal capping module referred to herein may relate to the corresponding position(s) of SEQ ID NO: 1. Accordingly, in some embodiments, the position(s) of the N-terminal capping module relate to the corresponding position(s) of SEQ ID NO: 1. In light of the high sequence similarity of SEQ ID NOs: 1 to 60, the respective positions of these sequences are well aligned and the position(s) of the N-terminal capping module referred to herein may similarly relate to the corresponding position(s) of one or more of SEQ ID NOs: 1 to 60. Accordingly, in some embodiments, the position(s) of the N-terminal capping module relate to the corresponding position(s) of any one of SEQ ID NOs: 1 to 60. In particular, in embodiments further defining the sequence of the N-terminal capping module by way of reference to one or more of SEQ ID NOs: 1 to 60, the position(s) of the N-terminal capping module may relate to the corresponding position(s) of the respective one or more sequence of SEQ ID NOs: 1 to 60 used to further define the sequence of the N-terminal capping module. For instance, for an ankyrin repeat domain having an N-terminal capping module with L at position 17 and comprising a sequence with at least 70% sequence identity to one or more of SEQ ID NOs: 2 to 9, position 17 may refer to the position corresponding to position 17 of SEQ ID NO: 1 or it may refer to the position corresponding to position 17 of the respective sequence of any one of SEQ ID NOs: 2 to 9 with which the N-terminal capping module shares said sequence identity. If an embodiment specifies, for instance, that the ankyrin repeat protein has an N-terminal capping module comprising a sequence with at least 70% sequence identity to an amino acid sequence selected from the group consisting of SEQ ID NOs: 2 to 9 and L at the position corresponding to position 17 of such amino acid sequence selected from the group consisting of SEQ ID NOs: 2 to 9 with which it shares said sequence identity, it can be determined for any given ankyrin repeat domain sequence whether it fulfills these conditions by aligning it to each of SEQ ID NOs: 2 to 9 to verify whether it has at least 70% sequence identity to one of these sequences and also L at the position corresponding to position 17 of such sequence. The “position(s)” of an internal ankyrin repeat, for instance the one that is adjacent to the N-terminal capping module, referred to herein may relate to the corresponding position(s) of SEQ ID NO: 61, which is the archetypal internal ankyrin repeat of designed ankyrin repeat proteins that remains commonly used in scientific studies (Binz, 2003, loc. cit.). Accordingly, in some embodiments, the position(s) of an internal ankyrin repeat, e.g., the one that is adjacent to the N-terminal capping module, relate to the corresponding position(s) of SEQ ID NO: 61. In light of the high sequence similarity of SEQ ID NOs: 61 to 76, the respective positions of these sequences are well aligned and the position(s) of the internal ankyrin repeat, e.g., the one that is adjacent to the N-terminal capping module, referred to herein may similarly relate to the corresponding position(s) of one or more of SEQ ID NOs: 61 to 76. Accordingly, in some embodiments, the position(s) of the internal ankyrin repeat, e.g., the one that is adjacent to the N-terminal capping module, relate to the corresponding position(s) of any one of SEQ ID NOs: 61 to 76. In particular, in embodiments further defining the sequence of the internal ankyrin repeat, e.g., the one that is adjacent to the N-terminal capping module, by way of reference to one or more of SEQ ID NOs: 61 to 76, the position(s) of the internal ankyrin repeat may relate to the corresponding position(s) of the respective one or more sequence of SEQ ID NOs: 61 to 76 used to further define the sequence of the internal ankyrin repeat. For instance, for a protein comprising an ankyrin repeat domain having an internal ankyrin repeat that is adjacent to the N-terminal capping module with L at position 23 and comprising a sequence with at least 70% sequence identity to one or more of SEQ ID NOs: 65 to 68, position 23 may refer to the position corresponding to position 23 of SEQ ID NO: 61, which is V in SEQ ID NO: 61, or it may refer to the position corresponding to position 23 of the respective sequence of any one of SEQ ID NOs: 65 to 68 with which the internal ankyrin repeat shares said sequence identity. The “position(s)” of the C-terminal capping module referred to herein may relate to the corresponding position(s) of SEQ ID NO: 77, which is the archetypal C-terminal capping module of designed ankyrin repeat proteins that remains commonly used in scientific studies (Binz, 2003, loc. cit.). Accordingly, in some embodiments, the position(s) of the C-terminal capping module relate to the corresponding position(s) of SEQ ID NO: 77. In light of the high sequence similarity of SEQ ID NOs: 77 to 95, the respective positions of these sequences are well aligned and the position(s) of the C-terminal capping module referred to herein may similarly relate to the corresponding position(s) of one or more of SEQ ID NOs: 77 to 95. Accordingly, in some embodiments, the position(s) of the C-terminal capping module relate to the corresponding position(s) of any one of SEQ ID NOs: 77 to 95. In particular, in embodiments further defining the sequence of the C-terminal capping module by way of reference to one or more of SEQ ID NOs: 77 to 95, the position(s) of the C-terminal capping module may relate to the corresponding position(s) of the respective one or more sequence of SEQ ID NOs: 77 to 95 used to further define the sequence of the C-terminal capping module. For instance, for a protein comprising an ankyrin repeat domain having a C-terminal capping module with L at position 23 and comprising a sequence with at least 70% sequence identity to one or more of SEQ ID NOs: 80 to 84, position 23 may refer to the position corresponding to position 23 of SEQ ID NO: 77, which is I in SEQ ID NO: 77, or it may refer to the position corresponding to position 23 of the respective sequence of any one of SEQ ID NOs: 80 to 84 with which its shares said sequence identity. In some embodiments, the position(s) of the N-terminal capping module refer to the corresponding position(s) of SEQ ID NO: 1 and the position(s) of the internal ankyrin repeat(s), e.g., the one that is adjacent to the N-terminal capping module, refer to the corresponding position(s) of SEQ ID NO: 61. In some embodiments, the position(s) of the N-terminal capping module refer to the corresponding position(s) of SEQ ID NO: 1, the position(s) of the internal ankyrin repeat(s), e.g., the one that is adjacent to the N-terminal capping module, refer to the corresponding position(s) of SEQ ID NO: 61 and the position(s) of the C-terminal capping module refer to the corresponding position(s) of SEQ ID NO: 77. Furthermore, “corresponding” in this context means that the respective positions align in a sequence alignment. Alignment for purposes of determining which amino acid residue corresponds to which position of a specific sequence can be achieved in various ways, as is further described above.

The term “recombinant”, as used in reference to a protein, refers to a protein produced from a recombinant nucleic acid. A “recombinant nucleic acid” refers to a nucleic acid molecule formed by laboratory methods of genetic recombination or gene synthesis.

The term “target”, as used, for instance, in conjunction with the specific binding property of an ankyrin repeat domain, refers to any substance or structure. It may refer to a single molecule, such as a protein, peptide, small-molecule or sugar, as well as complexed molecules, such as interacting proteins or proteins binding to non-proteinaceous compounds. It may also refer to more macromolecular structures, such as cells, tissues, viruses or bacteria.

The terms “treating” or “treatment” of a disease, condition or symptom refers to obtaining therapeutic and/or prophylactic benefit, including alleviating, ablating, ameliorating, or preventing a disease, condition or symptom, preventing additional symptoms, ameliorating or preventing the underlying metabolic causes of symptoms, inhibiting the disease or condition, e.g., arresting or slowing down the development of the disease or condition, relieving the disease or condition, causing regression of the disease or condition, relieving a condition caused by the disease or condition, or stopping the symptoms of the disease or condition.

### DETAILED DESCRIPTION OF THE INVENTION

The protein of the invention comprises an ankyrin repeat domain that has a mutation at position 17 of the N-terminal capping module. In some embodiments, the amino acid residue at position 17 of the N-terminal capping module is selected from the group consisting of L, I and V. In some embodiments, the amino acid residue at position 17 of the N-terminal capping module is L.

In some embodiments, the ankyrin repeat domain of the protein of the invention has improved properties, which may include improved thermostability, improved storage stability, improved thermodynamic stability (defined as the difference in free energy between the folded and unfolded states), improved folding and/or refolding properties (such as a higher fraction of refolded ankyrin repeat domains after thermal denaturation), reduced aggregation propensity and lower in vivo immunogenicity risk. Thus, in some embodiments, the protein of the invention comprises an ankyrin repeat domain that has a mutation at position 17 of the N-terminal capping module, e.g., an amino acid residue selected from the group consisting of L, I and V, and an improved property, such as an improved thermostability, as compared to a reference ankyrin repeat domain having the same sequence except for said position 17, which is, e.g., E in the reference ankyrin repeat domain.

In some embodiments, the ankyrin repeat domain has further mutations apart from the mutation at position 17 of the N-terminal capping module.

In some embodiments, the ankyrin repeat domain has a further mutation in the N-terminal capping module that is selected from the following amino acid residues:

TABLE 1

| Position | Amino acid residue |
|---|---|
| 1 | A, E, N, Q, G, S, T, K, D, R, H, C |
| 2 | E, L, Q, M, K, R, C |
| 3 | G, D, S, A, C |
| 4 | A, R, T, S, N, Q, K, D, E, H, C |
| 5 | A, R, T, S, N, Q, K, D, E, H, C |
| 6 | A, L, N, S, D, C |
| 7 | L, I, V, A, N, T, S, D, C |
| 8 | E, D, Q, A, N, S, T, K, R, H, C |
| 9 | A |
| 10 | V, S, A, C |
| 11 | L, S, Q, K, R, A, H, D, E, C, T, N, F, W, |
| | Y |
| 12 | K, R, A, T, S, N, Q, D, E, H, C |
| 13 | G, C |
| 14 | N, S, T, A, D, E, K, Q, R, H, C |
| 15 | M, I, T, A, L, V, S, N, D, Q, K, R, E, C |
| 16 | D, A, N, Q, S, T, K, E, R, H, C |
| 18 | T, A, S, I, L, V, C |
| 19 | R, E, D, K, A, N, Q, S, T, H, C |
| 20 | N, K, R, T, S, E, Q, A, D, H, C, I, V |
| 21 | N, S, L, A, C |
| 22 | I, A, V, M, T, L, S, N, C |
| 23 | R, S, Q, K, N, A, E, D, H, C |
| 24 | A, H, K, R, L, I, V, C, G |
| 25 | G |
| 26 | A |
| 27 | N, D, C |
| 28 | T, V, S, P, A, C |
| 29 | D, N, C |
| 30 | A, C |

In some embodiments, the N-terminal capping module further has an amino acid residue of Table 1 in one or more of the respective position(s). In some embodiments, the amino acid residue at one or more position(s) of the N-terminal capping module is selected from the group consisting of the amino acid residues shown for the respective position(s) in Table 1.

In some embodiments, the N-terminal capping module further has an amino acid residue selected from the group consisting of G, D and A at position 3. In some embodiments, the N-terminal capping module has G at position 3. In some embodiments, the N-terminal capping module has D at position 3. In some embodiments, the N-terminal capping module has A at position 3.

In some embodiments, the N-terminal capping module further has an amino acid residue selected from the group consisting of E, Q, K and A at position 8. In some embodiments, the N-terminal capping module has E or Q at position 8. In some embodiments, the N-terminal capping module has E at position 8. In some embodiments, the N-terminal capping module has Q at position 8. In some embodiments, the N-terminal capping module has A at position 8.

In some embodiments, the N-terminal capping module further has an amino acid residue selected from the group consisting of L, S, Q, K, R, A, H, D and E at position 11. In some embodiments, the N-terminal capping module has an amino acid residue selected from the group consisting of K, E, Q, A and L at position 11. In some embodiments, the N-terminal capping module has an amino acid residue selected from the group consisting of K, E, A and L at position 11. In some embodiments, the N-terminal capping module has E or A at position 11. In some embodiments, the N-terminal capping module has A at position 11. In some embodiments, the N-terminal capping module has E at position 11.

In some embodiments, the N-terminal capping module further has an amino acid residue selected from the group consisting of I, T, A, V, L and M at position 15. In particular, L, I and V at position 15 of the N-terminal capping module were found to combine well with L or I at position 23 of the internal ankyrin repeat that is adjacent to the N-terminal capping module. Thus, in some embodiments, the N-terminal capping module has an amino acid residue selected from the group consisting of L, I and V at position 15 and the internal ankyrin repeat that is adjacent to the N-terminal capping module has L or I at position 23.

In some embodiments, the N-terminal capping module further has an amino acid residue selected from the group consisting of I, V and L at position 15. In some embodiments, the N-terminal capping module has I or V at position 15. In some embodiments, the N-terminal capping module has I at position 15. In some embodiments, the N-terminal capping module has V at position 15. In some embodiments, the N-terminal capping module has L at position 15.

In some embodiments, the internal ankyrin repeat that is adjacent to the N-terminal capping module has L at position 23 and the N-terminal capping module further has I at position 15. In some embodiments, the internal ankyrin repeat that is adjacent to the N-terminal capping module has L at position 23 and the N-terminal capping module further has L at position 15. In some embodiments, the internal ankyrin repeat that is adjacent to the N-terminal capping module has L at position 23 and the N-terminal capping module further has V at position 15. In some embodiments, the internal ankyrin repeat that is adjacent to the N-terminal capping module has L at position 23 and the N-terminal capping module further has T at position 15. In some embodiments, the internal ankyrin repeat that is adjacent to the N-terminal capping module has L at position 23 and the N-terminal capping module further has A at position 15. In some embodiments, the internal ankyrin repeat that is adjacent to the N-terminal capping module has L at position 23 and the N-terminal capping module further has M at position 15. In some embodiments, the internal ankyrin repeat that is adjacent to the N-terminal capping module has I at position 23 and the N-terminal capping module further has I at position 15. In some embodiments, the internal ankyrin repeat that is adjacent to the N-terminal capping module has I at position 23 and the N-terminal capping module further has L at position 15. In some embodiments, the internal ankyrin repeat that is adjacent to the N-terminal capping module has I at position 23 and the N-terminal capping module further has V at position 15. In some embodiments, the internal ankyrin repeat that is adjacent to the N-terminal capping module has I at position 23 and the N-terminal capping module further has T at position 15. In some embodiments, the internal ankyrin repeat that is adjacent to the N-terminal capping module has I at position 23 and the N-terminal capping module further has A at position 15. In some embodiments, the internal ankyrin repeat that is adjacent to the N-terminal capping module has I at position 23 and the N-terminal capping module further has M at position 15. In some embodiments, the internal ankyrin repeat that is adjacent to the N-terminal capping module has L or I at position 23 and the N-terminal capping module further has I at position 15.

In some embodiments, the N-terminal capping module further has an amino acid residue selected from the group consisting of D, E and Q at position 16. In some embodiments, the N-terminal capping module has D at position 16. In some embodiments, the N-terminal capping module has E at position 16. In some embodiments, the N-terminal capping module has Q at position 16.

In some embodiments, the N-terminal capping module further has an amino acid residue selected from the group consisting of R, E, D, K, A, N, Q, S, T, H and C at position 19. In some embodiments, the N-terminal capping module has R at position 19. In some embodiments, the N-terminal capping module has K at position 19.

In some embodiments, the N-terminal capping module further has an amino acid residue selected from the group consisting of Q, K, E and I at position 20. In some embodiments, the N-terminal capping module has Q at position 20. In some embodiments, the N-terminal capping module has K at position 20. In some embodiments, the N-terminal capping module has I at position 20. In some embodiments, the N-terminal capping module has E at position 20.

In some embodiments, the internal ankyrin repeat that is adjacent to the N-terminal capping module has L or I at position 23 and the N-terminal capping module further has I at position 15 and Q at position 20. In some embodiments, the internal ankyrin repeat that is adjacent to the N-terminal capping module has L or I at position 23 and the N-terminal capping module further has I at position 15 and K at position 20. In some embodiments, the internal ankyrin repeat that is adjacent to the N-terminal capping module has L or I at position 23 and the N-terminal capping module further has I at position 15 and I at position 20.

In some embodiments, the N-terminal capping module further has L at position 2. In some embodiments, the N-terminal capping module further has L at position 24. In some embodiments, the N-terminal capping module further has L at position 2 and L at position 24.

In some embodiments, the N-terminal capping module further has an amino acid residue selected from the group consisting of L, V, I and A at position 22. In some embodiments, the N-terminal capping module has an amino acid residue selected from the group consisting of L, V and I at position 22. In some embodiments, the N-terminal capping module has L at position 22. In some embodiments, the N-terminal capping module has V at position 22. In some embodiments, the N-terminal capping module has I at position 22. In some embodiments, the N-terminal capping module has A at position 22.

In some embodiments, the internal ankyrin repeat that is adjacent to the N-terminal capping module has L at position 23 and the N-terminal capping module further has I at position 22. In some embodiments, the internal ankyrin repeat that is adjacent to the N-terminal capping module has L at position 23 and the N-terminal capping module further has L at position 22. In some embodiments, the internal ankyrin repeat that is adjacent to the N-terminal capping module has L at position 23 and the N-terminal capping module further has V at position 22. In some embodiments, the internal ankyrin repeat that is adjacent to the N-terminal capping module has I at position 23 and the N-terminal capping module further has I at position 22. In some embodiments, the internal ankyrin repeat that is adjacent to the N-terminal capping module has I at position 23 and the N-terminal capping module further has L at position 22. In some embodiments, the internal ankyrin repeat that is adjacent to the N-terminal capping module has I at position 23 and the N-terminal capping module further has V at position 22.

In some embodiments, the N-terminal capping module further has an amino acid residue selected from the group consisting of I, T, A, V, L and M at position 15 and an amino acid residue selected from the group consisting of L, V and I at position 22.

In some embodiments, the N-terminal capping module further has L at position 15 and I at position 22. In some embodiments, the N-terminal capping module further has M at position 15 and I at position 22. In some embodiments, the N-terminal capping module further has T at position 15 and I at position 22. In some embodiments, the N-terminal capping module further has I at position 15 and I at position 22. In some embodiments, the N-terminal capping module further has A at position 15 and I at position 22. In some embodiments, the N-terminal capping module further has V at position 15 and I at position 22.

In some embodiments, the N-terminal capping module further has L at position 15 and L at position 22. In some embodiments, the N-terminal capping module further has M at position 15 and L at position 22. In some embodiments, the N-terminal capping module further has T at position 15 and L at position 22. In some embodiments, the N-terminal capping module further has I at position 15 and L at position 22. In some embodiments, the N-terminal capping module further has A at position 15 and L at position 22. In some embodiments, the N-terminal capping module further has V at position 15 and L at position 22.

In some embodiments, the N-terminal capping module further has L at position 15 and V at position 22. In some embodiments, the N-terminal capping module further has M at position 15 and V at position 22. In some embodiments, the N-terminal capping module further has T at position 15 and V at position 22. In some embodiments, the N-terminal capping module further has I at position 15 and V at position 22. In some embodiments, the N-terminal capping module further has A at position 15 and V at position 22. In some embodiments, the N-terminal capping module further has V at position 15 and V at position 22.

In some embodiments, the N-terminal capping module further has an amino acid residue selected from the group consisting of R, S, Q, K, N, A, E, D, H, C at position 23. In some embodiments, the N-terminal capping module has E at position 23. In some embodiments, the N-terminal capping module has A at position 23. In some embodiments, the N-terminal capping module has K at position 23.

In some embodiments, the N-terminal capping module further has an amino acid residue selected from the group consisting of I, T, A, V, L and M at position 15, an amino acid residue selected from the group consisting of R and K at position 19, and an amino acid residue selected from the group consisting of L, V and I at position 22. In some embodiments, the N-terminal capping module has an amino acid residue selected from the group consisting of I, T, A, V, L and M at position 15, the amino acid residue R at position 19 and an amino acid residue selected from the group consisting of L, V and I at position 22. In some embodiments, the N-terminal capping module has an amino acid residue selected from the group consisting of I, T, A, V, L and M at position 15, the amino acid residue K at position 19 and an amino acid residue selected from the group consisting of L, V and I at position 22.

In some embodiments, the N-terminal capping module further has an amino acid residue selected from the group consisting of I, T, A, V, L and M at position 15, an amino acid residue selected from the group consisting of L, V and I at position 22, and an amino acid residue selected from the group consisting of A and K at position 23. In some embodiments, the N-terminal capping module has an amino acid residue selected from the group consisting of I, T, A, V, L and M at position 15, an amino acid residue selected from the group consisting of L, V and I at position 22, and the amino acid residue A at position 23. In some embodiments, the N-terminal capping module has an amino acid residue selected from the group consisting of I, T, A, V, L and M at position 15, an amino acid residue selected from the group consisting of L, V and I at position 22, and the amino acid residue K at position 23.

In some embodiments, the N-terminal capping module further has an amino acid residue selected from the group consisting of I, T, A, V, L and M at position 15, an amino acid residue selected from the group consisting of R and K at position 19, an amino acid residue selected from the group consisting of L, V and I at position 22, and an amino acid residue selected from the group consisting of A and K at position 23. In some embodiments, the N-terminal capping module has an amino acid residue selected from the group consisting of I, T, A, V, L and M at position 15, the amino acid residue R at position 19, an amino acid residue selected from the group consisting of L, V and I at position 22, and the amino acid residue K at position 23. In some embodiments, the N-terminal capping module has an amino acid residue selected from the group consisting of I, T, A, V, L and M at position 15, the amino acid residue K at position 19, an amino acid residue selected from the group consisting of L, V and I at position 22, and the amino acid residue K at position 23. In some embodiments, the N-terminal capping module has an amino acid residue selected from the group consisting of I, T, A, V, L and M at position 15, the amino acid residue R at position 19, an amino acid residue selected from the group consisting of L, V and I at position 22, and the amino acid residue A at position 23. In some embodiments, the N-terminal capping module has an amino acid residue selected from the group consisting of I, T, A, V, L and M at position 15, the amino acid residue K at position 19, an amino acid residue selected from the group consisting of L, V and I at position 22, and the amino acid residue A at position 23.

In some embodiments, the N-terminal capping module further has an amino acid residue selected from the group consisting of E and A at position 11, an amino acid residue selected from the group consisting of I, T, A, V, L and M at position 15, an amino acid residue selected from the group consisting of R and K at position 19, an amino acid residue selected from the group consisting of L, V and I at position 22, and an amino acid residue selected from the group consisting of A and K at position 23.

In some embodiments, the N-terminal capping module further has an amino acid residue selected from the group consisting of R and K at position 19 and an amino acid residue selected from the group consisting of A and K at position 23. In some embodiments, the N-terminal capping module has R at position 19 and A at position 23. In some embodiments, the N-terminal capping module has K at position 19 and A at position 23. In some embodiments, the N-terminal capping module has R at position 19 and K at position 23. In some embodiments, the N-terminal capping module has K at position 19 and K at position 23.

In some embodiments, the N-terminal capping module further has L at position 24. In some embodiments, the internal ankyrin repeat that is adjacent to the N-terminal capping module has L at position 23 and the N-terminal capping module has L at position 24. In some embodiments, the internal ankyrin repeat that is adjacent to the N-terminal capping module has I at position 23 and the N-terminal capping module has L at position 24. In some embodiments, the N-terminal capping module has an amino acid residue selected from the group consisting of I, T, A, V, L and M at position 15 and L at position 24. In some embodiments, the N-terminal capping module has an amino acid residue selected from the group consisting of L, V and I at position 22 and L at position 24. In some embodiments, the N-terminal capping module has an amino acid residue selected from the group consisting of I, T, A, V, L and M at position 15, an amino acid residue selected from the group consisting of L, V and I at position 22 and L at position 24. In some embodiments, the internal ankyrin repeat that is adjacent to the N-terminal capping module has L or I at position 23 and the N-terminal capping module has I at position 15 and L at position 24. In some embodiments, the internal ankyrin repeat that is adjacent to the N-terminal capping module has L or I at position 23 and the N-terminal capping module has L at position 24.

In some embodiments, the N-terminal capping module further has the amino acid sequence (R/K)(I/E/Q/K)L(L/I/M)(A/K)(A/L) at positions 19 to 24, wherein the amino acid residue at the positions 19, 20, 22, 23 and 24 is selected from the group consisting of the amino acid residues shown in the respective parentheses. In some embodiments, the N-terminal capping module has one of the amino acid residues indicated for the respective positions in Table 1 at positions 19 to 24. In some embodiments, the N-terminal capping module has an amino acid sequence at positions 19 to 24 selected from the group consisting of: RELLKA, RILLKA, ROLLKA, RKLLKA, RILMAL, ROLMAL, RKLMAL, RELLKL, RILLKL, ROLLKL, RKLLKL, RELIKL, RILIKL, ROLIKL, RKLIKL, RELLAL, RILLAL, ROLLAL, RKLLAL, RELIAL, RILIAL, ROLIAL, RKLIAL, KILMAL, KOLMAL, KKLMAL, KELLKL, KILLKL, KOLLKL, KKLLKL, KELIKL, KILIKL, KOLIKL, KKLIKL, KELLAL, KILLAL, KOLLAL, KKLLAL, KELIAL, KILIAL, KQLIAL and KKLIAL. In some embodiments, the N-terminal capping module has the amino acid sequence KELIAL or KKLIAL at positions 19 to 24. In some embodiments, the internal ankyrin repeat that is adjacent to the N-terminal capping module has L or I at position 23 and the N-terminal capping module has I at position 15 and the amino acid sequence KELIAL or KKLIAL at positions 19 to 24.

In some embodiments, the N-terminal capping module does not comprise the amino acid sequence TPLH.

In some embodiments, the ankyrin repeat domain of the protein of the invention has an improved thermostability, such as a higher melting temperature and/or a higher fraction of refolded ankyrin repeat domains after thermal denaturation, as compared to a reference ankyrin repeat domain having the same amino acid sequence except for the mutation at position 17 of the N-terminal capping module. In some embodiments having one or more of the mutation(s) specified herein in addition to the mutation at position 17 of the N-terminal capping module, the ankyrin repeat domain of the protein of the invention has an improved thermostability, such as a higher melting temperature and/or a higher fraction of refolded ankyrin repeat domains after thermal denaturation, as compared to a reference ankyrin repeat domain having the same amino acid sequence except for the mutation at position 17 of the N-terminal capping module and/or as compared to a reference ankyrin repeat domain having the same amino acid sequence except for the one or more additional mutation(s) as specified herein and/or as compared to a reference ankyrin repeat domain having the same amino acid sequence except for the mutation at position 17 of the N-terminal capping module and except for the one or more additional mutation(s) as specified herein.

In some embodiments, the reference ankyrin repeat domain not having the mutation at position 17 of the N-terminal capping module has an amino acid residue selected from the naturally occurring amino acid residues other than L, I and V at this position, such as an amino acid residue selected from the group consisting of A, N, Q, S, T, K, E, R, H and C. In some embodiments, the reference ankyrin repeat domain not having one or more of the mutation(s) in the N-terminal capping module as specified herein has an amino acid residue found in the corresponding position(s) of SEQ ID NO: 42 or SEQ ID NO: 43. For instance, in case of a mutation at position 17 of the N-terminal capping module, the amino acid residue at corresponding position 17 of the reference ankyrin repeat domain can be E (see SEQ ID NOs: 42 and 43). Similarly, in case of a mutation at position 19 of the N-terminal capping module, the amino acid residue at corresponding position 19 of the reference ankyrin repeat domain can be R (see SEQ ID NOs: 42 and 43). Similarly, in case of a mutation at position 20 of the N-terminal capping module, the amino acid residue at corresponding position 20 of the reference ankyrin repeat domain can be I or E (see SEQ ID NOs: 42 and 43, respectively). Similarly, in case of a mutation at position 22 of the N-terminal capping module, the amino acid residue at corresponding position 22 of the reference ankyrin repeat domain can be M (see SEQ ID NO: 42). Similarly, in case of a mutation at position 24 of the N-terminal capping module, the amino acid residue at corresponding position 24 of the reference ankyrin repeat domain can be A (see SEQ ID NO: 43). In some embodiments, the reference ankyrin repeat domain not having one or more of the mutation(s) in the internal ankyrin repeat(s) as specified herein has an amino acid residue found in the corresponding position(s) of SEQ ID NO: 61. In some embodiments, the reference ankyrin repeat domain not having one or more of the mutation(s) in the C-terminal capping module as specified herein has an amino acid residue found in the corresponding position(s) of SEQ ID NO: 77.

In some embodiments, the ankyrin repeat domain of the protein of the invention (with or without additional mutations as specified herein) has an improved thermostability, such as a higher melting temperature and/or a higher fraction of refolded ankyrin repeat domains after thermal denaturation, as compared to a reference ankyrin repeat domain having the same amino acid sequence except for position 17 of the N-terminal capping module, which is E in the reference ankyrin repeat domain. In some embodiments, the ankyrin repeat domain of the protein of the invention additionally has a mutation at position 15 of the N-terminal capping module, e.g., an amino acid residue selected from the group consisting of L, I and V, and the ankyrin repeat domain has an improved thermostability, such as a higher melting temperature and/or a higher fraction of refolded ankyrin repeat domains after thermal denaturation, as compared to a first reference ankyrin repeat domain having the same amino acid sequence except for the mutation at position 17 of the N-terminal capping module, which is E in the first reference ankyrin repeat domain, and/or as compared to a second reference ankyrin repeat domain having the same amino acid sequence except for position 15 of the N-terminal capping module, which is D in the second reference ankyrin repeat domain, and/or as compared to a third reference ankyrin repeat domain having the same amino acid sequence except for the mutation at position 17 of the N-terminal capping module, which is E in the third reference ankyrin repeat domain, and except for position 15 of the N-terminal capping module, which is D in the third reference ankyrin repeat domain. In some embodiments, the ankyrin repeat domain of the protein of the invention additionally has a mutation at position 23 of the internal ankyrin repeat that is adjacent to the N-terminal capping module, e.g., an amino acid residue selected from the group consisting of L and I, and the ankyrin repeat domain has an improved thermostability, such as a higher melting temperature and/or a higher fraction of refolded ankyrin repeat domains after thermal denaturation, as compared to a first reference ankyrin repeat domain having the same amino acid sequence except for position 17 of the N-terminal capping module, which is E in the first reference ankyrin repeat domain, and/or as compared to a second reference ankyrin repeat domain having the same amino acid sequence except for the mutation at position 23 of the internal ankyrin repeat that is adjacent to the N-terminal capping module, which is V in the second reference ankyrin repeat domain, and/or as compared to a third reference ankyrin repeat domain having the same amino acid sequence except for the mutation at position 23 of the internal ankyrin repeat that is adjacent to the N-terminal capping module, which is V in the third reference ankyrin repeat domain, and except for position 17 of the N-terminal capping module, which is E in the third reference ankyrin repeat domain. In some embodiments, the ankyrin repeat domain of the protein of the invention additionally has a mutation at position 20 of the N-terminal capping module, e.g., an amino acid residue selected from the group consisting of Q or K and the ankyrin repeat domain has an improved thermostability, such as a higher melting temperature and/or a higher fraction of refolded ankyrin repeat domains after thermal denaturation, as compared to a first reference ankyrin repeat domain having the same amino acid sequence except for the mutation at position 17 of the N-terminal capping module, which is E in the first reference ankyrin repeat domain, and/or as compared to a second reference ankyrin repeat domain having the same amino acid sequence except for position 20 of the N-terminal capping module, which is E or I in the second reference ankyrin repeat domain, and/or as compared to a third reference ankyrin repeat domain having the same amino acid sequence except for the mutation at position 17 of the N-terminal capping module, which is E in the third reference ankyrin repeat domain, and except for position 20 of the N-terminal capping module, which is E or I in the third reference ankyrin repeat domain. In some embodiments, the ankyrin repeat domain of the protein of the invention additionally has a mutation at position 22 of the N-terminal capping module, e.g., an amino acid residue selected from the group consisting of I and L, and the ankyrin repeat domain has an improved thermostability, such as a higher melting temperature and/or a higher fraction of refolded ankyrin repeat domains after thermal denaturation, as compared to a first reference ankyrin repeat domain having the same amino acid sequence except for the mutation at position 17 of the N-terminal capping module, which is E in the first reference ankyrin repeat domain, and/or as compared to a second reference ankyrin repeat domain having the same amino acid sequence except for position 22 of the N-terminal capping module, which is M in the second reference ankyrin repeat domain, and/or as compared to a third reference ankyrin repeat domain having the same amino acid sequence except for the mutation at position 17 of the N-terminal capping module, which is E in the third reference ankyrin repeat domain, and except for position 22 of the N-terminal capping module, which is M in the third reference ankyrin repeat domain. In some embodiments, the ankyrin repeat domain of the protein of the invention additionally has a mutation at position 24 of the N-terminal capping module, e.g., L, and the ankyrin repeat domain has an improved thermostability, such as a higher melting temperature and/or a higher fraction of refolded ankyrin repeat domains after thermal denaturation, as compared to a first reference ankyrin repeat domain having the same amino acid sequence except for the mutation at position 17 of the N-terminal capping module, which is E in the first reference ankyrin repeat domain, and/or as compared to a second reference ankyrin repeat domain having the same amino acid sequence except for position 24 of the N-terminal capping module, which is A in the second reference ankyrin repeat domain, and/or as compared to a third reference ankyrin repeat domain having the same amino acid sequence except for the mutation at position 17 of the N-terminal capping module, which is E in the third reference ankyrin repeat domain, and except for position 24 of the N-terminal capping module, which is A in the third reference ankyrin repeat domain.

In some embodiments, the ankyrin repeat domain of the protein of the invention additionally has one or more further mutation(s) as specified herein and the mutation at position 17 of the N-terminal capping module and the one or more further mutation(s) as specified herein at least additively increase thermostability of the ankyrin repeat domain. Such at least additively increased thermostability may be reflected, for instance, by an at least additively increased melting temperature or an at least additively increased fraction of refolded ankyrin repeat domains after thermal denaturation.

Methods for measuring the thermostability of a protein or a protein domain are well-known to the person skilled in the art. For instance, the thermostability can be measured by a thermal shift assay, differential scanning calorimetry and circular dichroism (CD). Another possible approach is to use differential scanning fluorimetry (e.g. Nielsen et al., 2007, Nat Protoc. 2, 9:2212-21). In this method, unfolding of the protein is measured with a fluorescent dye that binds to hydrophobic parts of the protein. As the protein unfolds, more hydrophobic parts become exposed causing an increase in fluorescence and vice versa. This method therefore allows to conveniently monitor the refolding properties of a protein and to determine its melting temperature, which corresponds to the midpoint of the fluorescence transition curve. The refolding properties and melting temperature of a protein can also be measured by CD spectroscopy, whereby the thermal melting curve of the protein is determined by measuring the CD signal at 222 nm. For purposes of measuring the thermostability, the protein may be dissolved in, e.g., PBS or HEPES buffer. For example, the thermostability of a helical protein, such as an ankyrin repeat domain, can be determined by measuring the CD signal of the protein at 222 nm while slowly heating the protein at a concentration of 0.01 mM in the buffer from 20° C. to 95° C. using a temperature ramp of, e.g., 0.8° C. or 1° C. per min. A denaturant, such as guanidinium chloride, may be added to the buffer, e.g., if measuring a protein that does not fully unfold at 95° C.

In some embodiments, the increase in melting temperature of the ankyrin repeat domain of the invention is at least 1° C., at least 2° C., at least 3° C., at least 4° C. or at least 5° C., as compared to the reference ankyrin repeat domain(s).

In some embodiments, the fraction of the refolded ankyrin repeat domains after thermal denaturation is at least 1%, at least 5%, at least 10% or at least 20% higher, as compared to the reference ankyrin repeat domain(s).

Unless specified, the sequence of the ankyrin repeat domain is not particularly limited. In particular, the ankyrin repeat domain allows for a large sequence variation while preserving the overall structure and function of the domain.

In some embodiments, the N-terminal capping module is derived from the GA-binding protein, e.g., the GA-binding domain having the sequence of chain B of the PDB entry 1AWC. N-terminal capping modules with sequences similar to the N-terminal capping module of the GA-binding protein capping module find reflection in the sequences of SEQ ID NOs: 1 to 60 and the N-terminal capping modules of the ankyrin repeat domains used in the examples. In some embodiments, the N-terminal capping module comprises an amino acid sequence that has at least 70%, at least 75%, at least 80%, at least 85%, at least 90%, at least 95%, or 100% sequence identity to an amino acid sequence selected from the group consisting of SEQ ID NOs: 1 to 60. In some embodiments, the N-terminal capping module comprises an amino acid sequence that has at least 70%, at least 75%, at least 80%, at least 85%, at least 90%, at least 95%, or 100% sequence identity to an amino acid sequence selected from the group consisting of SEQ ID NOs: 1 to 12. In some embodiments, the N-terminal capping module comprises an amino acid sequence selected from the group consisting of SEQ ID NOs: 1 to 12 with up to 9, up to 8, up to 7, up to 6, up to 5, up to 4, up to 3, up to 2, up to 1 or no modifications selected from substitutions, insertions and deletions outside position 17 of SEQ ID NOs: 1 to 12. In some embodiments, the combined number of deletions and insertions in these modifications is not more than 2 or not more than 1. In some embodiments, all modifications are substitutions. In some embodiments, the N-terminal capping module comprising any of the amino acid sequences or amino acid sequence variants of this paragraph excludes those variants of the N-terminal capping module comprising the amino acid sequence TPLH.

The N-terminal capping module may further comprise a sequence directly N-terminal to the amino acid sequences defined in SEQ ID NOs: 1 to 60 (or the sequence variants thereof defined herein). For instance, such sequence could be a dipeptide comprising amino acid residues selected from the group consisting of D, A, E, N, Q, S, T, K, R and H, such as the dipeptide GS, DA, EA, AA, AD, AE, NA, AN, PT, TP, AT or TA. For instance, G and S or D and A could be at positions −2 and −1 of the N-terminal capping module, respectively. For instance, DA could be directly N-terminal of SEQ ID NO: 2. DA could also be directly N-terminal of SEQ ID NO: 5 or of SEQ ID NO: 9. Other exemplary N-terminal capping modules having the amino acid residues GS or DA N-terminal are reflected by the ankyrin repeat domains of the examples. Such dipeptide sequence may serve as a linker to connect the ankyrin repeat domain to the further peptide sequence of the protein or as an extended alpha-helix of the N-terminal capping module.

In some embodiments, the internal ankyrin repeat(s) of the ankyrin repeat domain consist of 33 amino acid residues.

In some embodiments, the ankyrin repeat domain comprises an internal ankyrin repeat comprising an amino acid sequence that has at least 70%, at least 75%, at least 80%, at least 85%, at least 90%, at least 95%, or 100% sequence identity to an amino acid sequence selected from the group consisting of SEQ ID NOs: 61 to 76. In some embodiments, each internal ankyrin repeat (of the one or more internal ankyrin repeats) of the ankyrin repeat domain comprises an amino acid sequence that has at least 70%, at least 75%, at least 80%, at least 85%, at least 90%, at least 95%, or 100% sequence identity to an amino acid sequence selected from the group consisting of SEQ ID NOs: 61 to 76. In some embodiments, the internal ankyrin repeat adjacent to the N-terminal capping module of the ankyrin repeat domain comprises an amino acid sequence that has at least 70%, at least 75%, at least 80%, at least 85%, at least 90%, at least 95%, or 100% sequence identity to an amino acid sequence selected from the group consisting of SEQ ID NOs: 61 to 76.

In some embodiments, one or more internal ankyrin repeat(s), e.g., the internal ankyrin repeat adjacent to the N-terminal capping module, have an amino acid residue selected from the group consisting of I, V, A, S and L at position 11.

In some embodiments, one or more internal ankyrin repeat(s), e.g., the internal ankyrin repeat adjacent to the N-terminal capping module, have an amino acid residue selected from the group consisting of I, V and L at position 18.

In some embodiments, one or more internal ankyrin repeat(s), e.g., the internal ankyrin repeat adjacent to the N-terminal capping module, have an amino acid residue selected from the group consisting of E, K, Q and A at position 19.

In some embodiments, one or more internal ankyrin repeat(s), e.g., the internal ankyrin repeat adjacent to the N-terminal capping module, have an amino acid residue selected from the group consisting of I, V and L at position 23. In some embodiments, one or more internal ankyrin repeat(s), e.g., the internal ankyrin repeat adjacent to the N-terminal capping module, have an amino acid residue selected from the group consisting of I, V and L at position 18 and an amino acid residue selected from the group consisting of I, V and L at position 23. In some embodiments, one or more internal ankyrin repeat(s), e.g., the internal ankyrin repeat adjacent to the N-terminal capping module, have L at position 18 and L at position 23. In some embodiments, the ankyrin repeat domain comprises (at least) two internal ankyrin repeats, wherein the N-terminal internal ankyrin repeat of these two internal ankyrin repeats has an amino acid residue selected from the group consisting of I, V and L at position 18 and the C-terminal internal ankyrin repeat of these two internal ankyrin repeats has an amino acid residue selected from the group consisting of I and L at position 23. For instance, the N-terminal internal ankyrin repeat has I at position 18 and the C-terminal internal ankyrin repeat has I at position 23, the N-terminal internal ankyrin repeat has I at position 18 and the C-terminal internal ankyrin repeat has L at position 23, the N-terminal internal ankyrin repeat has V at position 18 and the C-terminal internal ankyrin repeat has I at position 23, the N-terminal internal ankyrin repeat has V at position 18 and the C-terminal internal ankyrin repeat has L at position 23, the N-terminal internal ankyrin repeat has L at position 18 and the C-terminal internal ankyrin repeat has I at position 23 or the N-terminal internal ankyrin repeat has L at position 18 and the C-terminal internal ankyrin repeat has L at position 23. In some embodiments, the ankyrin repeat domain has more than two, e.g., three, four, five or six internal ankyrin repeats, each having the aforementioned mutations at positions 18 and 23, respectively.

In some embodiments, one or more internal ankyrin repeat(s), e.g., the internal ankyrin repeat adjacent to the N-terminal capping module, have an amino acid residue selected from the group consisting of E, K, Q and A at position 26.

In some embodiments having more than one internal ankyrin repeat, the internal ankyrin repeats share a high degree of sequence identity. In some embodiments, the internal ankyrin repeats share at least 70%, at least 75%, at least 80%, at least 85%, at least 90% or at least 95% sequence identity.

In some embodiments, the C-terminal capping module comprises an amino acid sequence that has at least 70%, at least 75%, at least 80%, at least 85%, at least 90%, at least 95%, or 100% sequence identity to an amino acid sequence selected from the group consisting of SEQ ID NOs: 77 to 95.

In some embodiments, the C-terminal capping module has an amino acid residue selected from the group consisting of D, H and N at position 10.

In some embodiments, the C-terminal capping module has an amino acid residue selected from the group consisting of A, N, L and Q at position 14.

In some embodiments, the C-terminal capping module has an amino acid residue selected from the group consisting of E, K and Q at position 18.

In some embodiments, the C-terminal capping module has K or A at position 19.

In some embodiments, the C-terminal capping module has an amino acid residue selected from the group consisting of A, T and V at position 21.

In some embodiments, the C-terminal capping module has E or K at position 22.

In some embodiments, the C-terminal capping module has an amino acid residue selected from the group consisting of Q, I, V and L at position 25.

In some embodiments, the C-terminal capping module has an amino acid residue selected from the group consisting of K, E and Q at position 26.

In some embodiments, the internal ankyrin repeat that is adjacent to the C-terminal capping module has an amino acid residue selected from the group consisting of I, V and L at position 18. In some embodiments, the C-terminal capping module has an amino acid residue selected from the group consisting of I, V and L at position 23. In some embodiments, the internal ankyrin repeat that is adjacent to the C-terminal capping module has an amino acid residue selected from the group consisting of I, V and L at position 18 and the C-terminal capping module has an amino acid residue selected from the group consisting of I and L at position 23. For instance, the internal ankyrin repeat that is adjacent to the C-terminal capping module has I at position 18 and the C-terminal capping module has I at position 23, the internal ankyrin repeat that is adjacent to the C-terminal capping module has I at position 18 and the C-terminal capping module has L at position 23, the internal ankyrin repeat that is adjacent to the C-terminal capping module has V at position 18 and the C-terminal capping module has I at position 23, the internal ankyrin repeat that is adjacent to the C-terminal capping module has V at position 18 and the C-terminal capping module has L at position 23, the internal ankyrin repeat that is adjacent to the C-terminal capping module has L at position 18 and the C-terminal capping module has I at position 23 or the internal ankyrin repeat that is adjacent to the C-terminal capping module has L at position 18 and the C-terminal capping module has L at position 23.

In some embodiments, the N-terminal capping module comprises an amino acid sequence that has at least 70%, at least 75%, at least 80%, at least 85%, at least 90%, at least 95%, or 100% sequence identity to an amino acid sequence selected from the group consisting of SEQ ID NOs: 1 to 12 and the internal ankyrin repeat that is adjacent to the N-terminal capping module comprises an amino acid sequence that has at least 70%, at least 75%, at least 80%, at least 85%, at least 90%, at least 95%, or 100% sequence identity to an amino acid sequence selected from the group consisting of SEQ ID NOs: 61 to 76. In some embodiments, the internal ankyrin repeat that is adjacent to the N-terminal capping module and the N-terminal capping module of the above embodiments in this paragraph each have at least 75% sequence identity to the indicated sequences. In some embodiments, the internal ankyrin repeat that is adjacent to the N-terminal capping module and the N-terminal capping module of the above embodiments in this paragraph each have at least 80% sequence identity to the indicated sequences. In some embodiments, the internal ankyrin repeat that is adjacent to the N-terminal capping module and the N-terminal capping module of the above embodiments in this paragraph each have at least 85% sequence identity to the indicated sequences. In some embodiments, the internal ankyrin repeat that is adjacent to the N-terminal capping module and the N-terminal capping module of the above embodiments in this paragraph each have at least 90% sequence identity to the indicated sequences. In some embodiments, the internal ankyrin repeat that is adjacent to the N-terminal capping module and the N-terminal capping module of the above embodiments in this paragraph each have at least 95% sequence identity to the indicated sequences. In some embodiments, the internal ankyrin repeat that is adjacent to the N-terminal capping module and the N-terminal capping module of the above embodiments in this paragraph each have 100% sequence identity to the indicated sequences.

In some embodiments, the N-terminal capping module comprises an amino acid sequence that has at least 70%, at least 75%, at least 80%, at least 85%, at least 90%, at least 95%, or 100% sequence identity to an amino acid sequence selected from the group consisting of SEQ ID NOs: 1 to 12, each internal ankyrin repeat comprises an amino acid sequence that has at least 70%, at least 75%, at least 80%, at least 85%, at least 90%, at least 95%, or 100% sequence identity to an amino acid sequence selected from the group consisting of SEQ ID NOs: 61 to 76, and the C-terminal capping module comprises an amino acid sequence that has at least 70%, at least 75%, at least 80%, at least 85%, at least 90%, at least 95%, or 100% sequence identity to an amino acid sequence selected from the group consisting of SEQ ID NOs: 77 to 95. In some embodiments, the sequence identity to the above sequences of the N-terminal capping module, internal ankyrin repeat(s) and C-terminal capping module in this paragraph is at least 70%. In some embodiments, the sequence identity to the above sequences of the N-terminal capping module, internal ankyrin repeat(s) and C-terminal capping module in this paragraph is at least 75%. In some embodiments, the sequence identity to the above sequences of the N-terminal capping module, internal ankyrin repeat(s) and C-terminal capping module in this paragraph is at least 80%. In some embodiments, the sequence identity to the above sequences of the N-terminal capping module, internal ankyrin repeat(s) and C-terminal capping module in this paragraph is at least 85%. In some embodiments, the sequence identity to the above sequences of the N-terminal capping module, internal ankyrin repeat(s) and C-terminal capping module in this paragraph is at least 90%. In some embodiments, the sequence identity to the above sequences of the N-terminal capping module, internal ankyrin repeat(s) and C-terminal capping module in this paragraph is at least 95%. In some embodiments, the sequence identity to the above sequences of the N-terminal capping module, internal ankyrin repeat(s) and C-terminal capping module in this paragraph is 100%.

It is understood that for those embodiments having defined the N-terminal capping module, internal ankyrin repeat(s) and/or C-terminal capping module by a certain mutation(s), e.g., L, I or V at position 17 of the N-terminal capping module, as well as a minimal sequence identity to an amino acid sequence, both conditions need to be fulfilled. For instance, an N-terminal capping module having L at position 17 and at least 70% sequence identity to an amino acid sequence selected from the group consisting of SEQ ID NOs: 1 to 12, relates to those embodiments wherein the N-terminal capping module has L at position 17 and, at the same time, at least 70% sequence identity to one or more of SEQ ID NOs: 1 to 12.

In some embodiments, the sequence of the ankyrin repeat domain is modified to reduce kidney uptake of the ankyrin repeat domain. In some embodiments, such ankyrin repeat domain is fused to a drug or radioligand, e.g., for use as a drug conjugate or radiopharmaceutical, respectively. Uptake of radiolabeled peptides or proteins by the kidney is a problem because of nephrotoxicity. It is known that such kidney uptake can be mediated by positive or negative charges present on these polypeptides and can be reduced by the application of cationic amino acids (e.g., lysine) or anionic amino acids (e.g., in form of polyglutamic acids), respectively. The megalin/cubilin receptor complex present on the brush border of proximal tubular cells in the kidney is involved in such charge-mediated kidney uptake of polypeptides. Accordingly, in some embodiments, the surface charge of the ankyrin repeat domain is changed, e.g., by replacing negatively charged amino acid residues from the surface of the ankyrin repeat domain, such as glutamate or aspartate, with amino acid residues that are not negatively charged, such as hydrophilic (e.g., serine or threonine), hydrophobic (e.g., leucine or valine), aromatic (e.g., tyrosine) or positively charged amino acids (e.g., lysine or arginine). In some embodiments, the negatively charged amino acid residue(s) on the surface of the ankyrin repeat domain become replaced with one of Q, K or L. In some embodiments, only negatively charged amino acid residues on the surface of the ankyrin repeat domain that are framework residues become replaced. For instance, the N-terminal capping module reflected by SEQ ID NO: 43 has glutamate or aspartate on the surface of the ankyrin repeat at positions 1, 8, 15, 16, 17, 20 and 27 of which one, or more, or all may be replaced with an amino acid residue that is not negatively charged such as Q, K or L, in particular, Q. Similarly, in another example, the internal ankyrin repeat reflected by SEQ ID NO: 61 has glutamate or aspartate on the surface of the ankyrin repeat at positions 2, 19, 22 and 30 of which one, or more, or all may be replaced with an amino acid residue that is not negatively charged such as Q, K or L, in particular, Q. Similarly, in another example, the C-terminal capping module reflected by SEQ ID NO: 77 has glutamate or aspartate on the surface of the ankyrin repeat at positions 2, 10, 14, 18, 19 and 22 of which one, or more, or all may be replaced with an amino acid residue that is not negatively charged such as Q, K or L, in particular, Q. In some embodiments, up to 1, 2, 3, 4, 5, 6, 7, 8, 9, 10, 15, 20 or all negatively charged amino acids on the surface of the ankyrin repeat domain become replaced. Exemplary ankyrin repeat domains having reduced negative charge are provided in WO 2016/023898 A2. In some embodiments positively charged amino acids, such as lysine or arginine, are replaced with amino acid residues that are not positively charged. In some embodiments positively and negatively charged amino acids are replaced by amino acid residues that are not charged.

In some embodiments, the ankyrin repeat domain comprises an N-terminal capping module, one internal ankyrin repeat and a C-terminal capping module (such ankyrin repeat domain structure is also referred to as “N1C”). In some embodiments, the ankyrin repeat domain comprises an N-terminal capping module, multiple internal ankyrin repeats, such as 2, 3, 4 or 5 internal ankyrin repeats, and a C-terminal capping module. In one embodiment, the ankyrin repeat domain comprises an N-terminal capping module, 2 or 3 internal ankyrin repeats and a C-terminal capping module (such ankyrin repeat domain structure is also referred to as “N2C” or “N3C”, respectively). In one embodiment, the ankyrin repeat domain has a N2C structure. In another embodiment, the ankyrin repeat domain has a N3C structure.

In some embodiments, the protein of the invention is a recombinant protein or a DARPin.

In some embodiments, the ankyrin repeat domain of the protein of the invention specifically binds to a target. For instance, the ankyrin repeat domain may specifically bind to a mammalian serum albumin, such as human serum albumin. Exemplary ankyrin repeat domains specifically binding to human serum albumin are disclosed in WO 2012/069654 A1 and also found in ensovibep (see amino acid residues 1-126 and 149-274 of ensovibep, respectively, as defined, e.g., in Proposed INN List: 124; WHO Drug Information, Vol. 34, No. 4, 2020). In some embodiments, the target is a peptide-MHC complex, such as peptide-MHC complexes having a peptide derived from HBcAg, HBsAg, EBNA-1, EBNA-2, EBNA-3, LMP-1, LMP-2, NSP-1, NSP-2, NSP-4, NSP-5, NSP-6, E1, E2, HBx, MAGE-A1, MAGE-A3, MAGE-A4, NY-ESO-1, PRAME, CT83 or SSX2. In some embodiments, the target is an extracellular protein or protein on a cell surface, such as IL4, IL13, HGF, PDGF, VEGF, Her2, CD3, CD4, CD8, CD33, CD40, CD70, CD123, FAP or 4-1BB. In some embodiments, the target is an intracellular protein. In some embodiments, the target is a protein on the surface of a virus, such as the spike protein of SARS-CoV-2.

In some embodiments, the protein only comprises a single ankyrin repeat domain.

The protein may also comprise or be bound to one or more further moieties in addition to the ankyrin repeat domain having the N-terminal capping module with the mutation at position 17, such as a moiety binding to a target, a labeling moiety, a toxic moiety, a moiety improving the pharmacokinetics, a moiety providing effector functions, a moiety allowing for the purification of the protein, a moiety providing enzymatic activity or a vector moiety. In some embodiments, the further moiety binding to a target is another ankyrin repeat domain, an antibody or fragment thereof or a receptor protein. In some embodiments, the further moiety binding to a target is another ankyrin repeat domain. In some embodiments, the labeling moiety is a stable isotope, a mass tag or a fluorescent label. In some embodiments, the toxic moiety is a chemotherapeutic agent, such as an alkylating agent, an antimetabolite, a taxane, or an anthracycline. In some embodiments, the moiety improving pharmacokinetics is a polypeptide (e.g., as used for PASylation), polyethylene glycol (PEG), a mammalian serum albumin, an immunoglobulin, a Fc domain of an immunoglobulin or a moiety binding to mammalian serum albumin or to an immunoglobulin. In one embodiment, the protein further contains an ankyrin repeat domain binding to a mammalian serum albumin. In some embodiments, the further moiety providing effector functions is a Fc domain of an immunoglobulin. In some embodiments, the moiety allowing for the purification of the protein is a FLAG-tag, a GST-tag, an HA-tag, a Myc-tag, a His-tag or a Strep-tag. In some embodiments, the further moiety providing enzymatic or fluorescence activity is, e.g., beta-lactamase or green fluorescence protein, respectively. In some embodiments, the further moiety is a vector moiety, e.g., a viral vector, such as an adeno-associated viral vector, an adenoviral vector or a lentiviral vector, or a non-viral vector, such as a lipid nanoparticle (LNP) vector.

The further moiety may be proteinaceous or non-proteinaceous.

In some embodiments, the further moiety in addition to the ankyrin repeat domain having the N-terminal capping module with the mutation at position 17 is one or more additional ankyrin repeat domain(s). In some embodiments, one or more of the additional ankyrin repeat domain(s) is an ankyrin repeat domain of the invention and thus also has a mutation at position 17 of the N-terminal capping module, such as an amino acid residue selected from the group consisting of L, I and V. In some embodiments, none of the additional one or more ankyrin repeat domain(s) has an amino acid residue selected from the group consisting of L, I and V at position 17 of the N-terminal capping module. In some embodiments, all of the additional ankyrin repeat domain(s) are ankyrin repeat domains of the invention. In some embodiments, the protein of the invention comprises more than one, e.g., at least two, at least three, at least four, at least five, or at least six, ankyrin repeat domains having an amino acid residue selected from the group consisting of L, I and V at position 17 of the N-terminal capping module. In some embodiments, the protein of the invention comprises more than one, e.g., at least two, at least three, at least four, at least five, or at least six, ankyrin repeat domains. In some embodiments, the protein of the invention comprises more than one, e.g., at least two, at least three, at least four, at least five, or at least six, ankyrin repeat domains each corresponding to an ankyrin repeat domain of the invention. In some embodiments, the protein of the invention comprises only one ankyrin repeat domain.

In some embodiments, the protein of the invention is multivalent, i.e. it comprises multiple identical moieties binding to the same target, in particular multiple identical ankyrin repeat domains binding to the same target. In some embodiments, the protein is bivalent, trivalent, tetravalent, pentavalent or hexavalent. In some embodiments, the protein of the invention is multiparatopic, i.e. it comprises multiple different moieties binding to the same target, in particular multiple different ankyrin repeat domains binding to the same target. In some embodiments, the protein is biparatopic, triparatopic, tetraparatopic, pentaparatopic or hexaparatopic. In some embodiments, the protein of the invention is multispecific, i.e. it comprises multiple different moieties binding to different targets, in particular multiple different ankyrin repeat domains binding to different targets. In some embodiments, the protein is bispecific, trispecific, tetraspecific, pentaspecific or hexaspecific. In some embodiments, the multivalent, multiparatopic or multispecific protein has more than one ankyrin repeat domain of the invention. In some embodiments, the multivalent, multiparatopic or multispecific protein has ankyrin repeat domains that are all ankyrin repeat domains of the invention.

The various moieties of the protein, including said ankyrin repeat domain of the invention, may bind covalently and/or non-covalently to one another. The various moieties may bind covalently to one another, for instance, via a peptide linker or via a maleimide-containing crosslinker. Suitable peptide linkers include glycine-serine linkers and proline-threonine linkers. In some embodiments, the suitable peptide linker is a naturally found peptide linker, such as the IgG hinge region. In some embodiments, the peptide linkers have a length of 2 to 24 amino acid residues or 2 to 16 amino acid residues. Exemplary peptide linkers include the linkers of SEQ ID NOs: 96 to 98. The various moieties may also bind non-covalently to one another, for instance, via a multimerization moiety. In some embodiments, a multimerization moiety is an immunoglobulin heavy chain constant region, a leucine zipper or a free thiol which can form a disulfide bond with another free thiol.

In some embodiments, the protein comprises one or more additional ankyrin repeat domains as further moieties that are bound to the protein by a proline-threonine linker.

The ankyrin repeat domain of the invention may be derived from various methods, such as selection from a protein library, in silico design or by mutating an existing ankyrin repeat domain. Subsequently, the protein comprising the ankyrin repeat domain of the invention (and possibly one or more further moieties bound to it) may be expressed or synthesized by methods known in the art and, e.g., formulated as a pharmaceutical product.

Accordingly, in a further aspect, the present disclosure relates to a library of proteins comprising one or more proteins of the invention. In some embodiments, the protein library comprises at least 10³, at least 10⁵, at least 10⁷, at least 10⁹, at least 10¹⁰, at least 10¹¹, at least 10¹² or at least 10¹³ proteins, each protein comprising an ankyrin repeat domain, and the library comprising one or more proteins of the invention. In some embodiments, the protein library comprises at least 10³, at least 10⁵, at least 10⁷, at least 10⁹, at least 10¹⁰, at least 10¹¹, at least 10¹² or at least 10¹³ proteins of the invention. In some embodiments, the protein library comprises at least 10³, at least 10⁵, at least 10⁷, at least 10⁹, at least 10¹⁰, at least 10¹¹, at least 10¹² or at least 10¹³ proteins that differ in the amino acid sequence of their ankyrin repeat domain and the library comprising one or more proteins of the invention. In some embodiments, the protein library comprises at least 10³, at least 10⁵, at least 10⁷, at least 10⁹, at least 10¹⁰, at least 10¹¹, at least 10¹² or at least 10¹³ proteins of the invention that differ in the amino acid sequence of their ankyrin repeat domain. In some embodiments, substantially all proteins of the protein library differ in the amino acid sequence of their ankyrin repeat domain. In some embodiments, substantially all proteins of the protein library are proteins of the invention. In some embodiments, the protein library comprises at least one protein of the invention.

In some embodiments, the protein library comprises proteins having ankyrin repeat domains with different structures. For instance, the protein library may contain a mixture of proteins comprising N2C and N3C ankyrin repeat domains. In some embodiments, the structure of the ankyrin repeat domain is identical for all proteins of the library, e.g., the ankyrin repeat domain of all proteins is either exclusively of N2C structure or exclusively of N3C structure. In some embodiments, the ankyrin repeat domain of all proteins is of the N2C structure. In other embodiments, the ankyrin repeat domain of all proteins is of the N3C structure. In some embodiments, the proteins of the library each comprise a single ankyrin repeat domain only.

The sequence variability in the ankyrin repeat domains of the protein library may be brought about randomly, e.g., by error-prone PCR of the nucleic acid molecules encoding the proteins, or it may be obtained by rational design followed by, e.g., direct synthesis of the nucleic acid molecules encoding the proteins (“design approach”). In some embodiments, the variability is introduced by the design approach. In the design approach, variability of the amino acid sequence is introduced in one or more than one position of the ankyrin repeat domains. The variable positions that may be occupied by different amino acid residues are also referred to as “randomized positions”, whereas the positions that are always occupied by the same amino acid residue are referred to as “fixed positions”. In some embodiments, the randomized positions are those positions occupied by potential target interaction residues and/or the fixed positions are those positions occupied by framework residues. In some embodiments, one or more of the positions occupied by potential target interaction residues are randomized positions. In some embodiments, all positions occupied by potential target interaction residues are randomized positions. In some embodiments, one or more of the positions occupied by framework residues are fixed positions. In some embodiments, all positions occupied by framework residues are fixed positions.

In certain embodiments, there are corresponding fixed positions and randomized positions in the ankyrin repeat domain of the different proteins of the protein library. Due to the intended variability in the randomized positions, the amino acid residues in corresponding randomized position may differ, although there may also be identical amino acid residues in corresponding randomized positions for at least some of the proteins in the library (though, in such cases, the proteins will not necessarily have identical amino acid residues in each of their corresponding randomized positions). In some embodiments, the fixed positions and the randomized positions are the same for the ankyrin repeat domains of each protein of the protein library. In some embodiments of ankyrin repeat domains having multiple internal ankyrin repeats, the internal ankyrin repeats of each ankyrin repeat domain have different randomized and fixed positions. In some embodiments of ankyrin repeat domains having multiple internal ankyrin repeats, the internal ankyrin repeats of each ankyrin repeat domain have different randomized and fixed positions and the fixed positions and the randomized positions are the same for the ankyrin repeat domains of each protein of the protein library. In some embodiments of ankyrin repeat domains having multiple internal ankyrin repeats, the internal ankyrin repeats of each ankyrin repeat domain have the same randomized and fixed positions. In some embodiments of ankyrin repeat domains having multiple internal ankyrin repeats, the internal ankyrin repeats of each ankyrin repeat domain have the same randomized and fixed positions and the fixed positions and the randomized positions are the same for the ankyrin repeat domains of each protein of the protein library.

The randomized positions may show different degrees of variability, i.e. they may be occupied by different sets of amino acid residues. The “X” amino acid residues of SEQ ID NOs: 61 to 76 and 86 are such randomized positions and, in some embodiments, may each be occupied by any amino acid residue. In some embodiments, the degree of variability differs between randomized positions. In some embodiments, the amino acid residue in a randomized position is any of the naturally occurring amino acid residues. In some embodiments, the amino acid residue in all randomized positions is any of the naturally occurring amino acid residues. In some embodiments, one or more randomized position(s) are only occupied by a subset of the naturally occurring amino acid residues. Such subsets can be those having common physicochemical properties, such as sets of hydrophobic, hydrophilic, acidic, basic, aromatic, or aliphatic amino acid residues. Other subsets are those comprising all naturally occurring amino acid residues except for certain non-desired amino acid residues, such as sets not comprising C or P. In some embodiments, one or more randomized position(s) are only occupied by any naturally occurring amino acid residue other than (i) an amino acid residue selected from the group consisting of C, G, M and N if followed by a G amino acid residue and (ii) P. In some embodiments, one or more randomized position(s) are only occupied by any naturally occurring amino acid residue other than C or other than C, G and P. In yet other embodiments, the subsets comprise those amino acid residues that are found in the corresponding positions of naturally occurring ankyrin repeats.

In some embodiments, the proteins of the protein library share at least 70%, at least 75%, at least 80%, at least 85%, at least 90% or at least 95% sequence identity in the amino acid sequence of their ankyrin repeat domains.

The above protein library can serve to select those proteins of the library that have a predetermined property, i.e. a certain property of interest that may be found in the ankyrin repeat domain of one of the proteins of the protein library and that can be screened for. Such predetermined property may include the specific binding to a target, the activation or inhibition of a target, such as an enzyme, and the blocking of an interaction between two targets. In some embodiments, the predetermined property is the specific binding to a target. Preferably, the protein selected from the library is a protein of the invention.

In one embodiment, the present disclosure provides a method for selecting a protein comprising an ankyrin repeat domain of the invention that specifically binds to a target, comprising the following steps:
a) providing a library of proteins comprising one or more proteins of the invention; and
b) selecting a protein specifically binding to the target via said ankyrin repeat domain from the library.

In one embodiment, the present disclosure provides a method for selecting a protein comprising an ankyrin repeat domain of the invention that specifically binds to a target, comprising the following steps:
a) providing a library of proteins of the invention; and
b) selecting a protein specifically binding to the target via said ankyrin repeat domain from the library.

During the selection step b), the proteins can be selected using screening methods commonly known to the person skilled in the art, such as yeast display, protein fragment complementation assay, phage display or ribosome display. The protein may also be selected during selection step b) by screening the library of step a) in silico. In some embodiments, the proteins are selected in step b) using phage display or ribosome display.

As indicated above, the protein of the invention as found in the protein library or represented by the protein selected from the library comprises an ankyrin repeat domain with an N-terminal capping module having a mutation at position 17 of the N-terminal capping module, such as an amino acid residue selected from the group consisting of L, I and V, and may have one or more further mutations in the ankyrin repeat domain as specified herein. In some embodiments, the N-terminal capping module of the ankyrin repeat domain comprises an amino acid sequence that has at least 70%, at least 75%, at least 80%, at least 85%, at least 90%, at least 95%, or 100% sequence identity to an amino acid sequence selected from the group consisting of SEQ ID NOs: 1 to 12. In some embodiments, the thermostability of the ankyrin repeat domain is improved in comparison to a reference ankyrin repeat domain having the same amino acid sequence except for the amino acid residue at position 17 of the N-terminal capping module, which is, e.g., E in the reference ankyrin repeat domain.

After the selection of a protein, the protein can be further modified, mutated and/or optimized by methods commonly known in the art.

For instance, amino acid sequence variants of the protein can be generated, e.g., by subjecting the nucleic acid encoding the selected protein to physical or chemical mutagens, copying said nucleic acid by error-prone PCR, using said nucleic acid for DNA shuffling or random chimeragenesis (Neylon C., Nucleic Acids Res., 32(4), 1448-1459, 2004). The protein library of such amino acid sequence variants may then again be subjected to the above selection step b) in order to select the variant(s) having the predetermined property. In some embodiments, there may be several rounds of selection under selection step b) under different conditions (with or without generating amino acid sequence variants of the protein(s) before each round).

The protein selected in step b) above may also be selectively mutated. For instance, one or more cysteine residues may be introduced, the thiol group(s) of which can then react with maleimide cross-linkers. Similarly, certain non-desirable amino acid residues may be removed, for instance, cysteines, which are prone to oxidations. Also, amino acid residues may be selectively mutated after analysis of the crystal structure so that the protein structure better fits to the target.

The protein selected in step b) may also become modified with one or more further moieties as outlined above for the protein of the invention. In one embodiment, the protein selected in step b) is modified with one or more further ankyrin repeat domains.

In one embodiment, the present disclosure provides a method of modifying a protein comprising an ankyrin repeat domain that does not have one or more mutations specified herein, e.g., one that does not have an amino acid residue selected from the group consisting of L, I and V at position 17 of the N-terminal capping module, by replacing one or more amino acid residues to result in a protein of the invention. By modifying an ankyrin repeat domain in this way, the favorable properties of the ankyrin repeat domain of the invention disclosed herein may be transferred to the ankyrin repeat domain of the thus obtained protein. In some embodiments, the amino acid residue at position 17 of the N-terminal capping module is replaced alone. In other embodiments, the amino acid residue at position 17 of the N-terminal capping module is replaced together with one or more other amino acid residues, e.g., those disclosed herein. In some embodiments, one or more of the further mutations in the N-terminal capping module referred to above are also introduced by replacing the amino acid residue(s) at the corresponding position(s). In some embodiments, the entire N-terminal capping module may be replaced.

Thus, in one embodiment, the present disclosure provides a method of preparing a protein or a method of improving the thermostability of an ankyrin repeat domain comprising the following steps:
a) selecting a protein comprising an ankyrin repeat domain with an N-terminal capping module that has none of L, I and V at position 17; and
b) replacing one or more amino acid residues of the protein to result in a protein of the invention.

In one embodiment, the present disclosure provides a method of preparing a protein or a method of improving the thermostability of an ankyrin repeat domain comprising the following steps:
a) selecting a protein comprising an ankyrin repeat domain with an N-terminal capping module that has none of L and I at position 17; and
b) replacing one or more amino acid residues of the protein to result in a protein of the invention comprising an ankyrin repeat domain with an N-terminal capping module having L or I at position 17.

In one embodiment, the present disclosure provides a method of preparing a protein or a method of improving the thermostability of an ankyrin repeat domain comprising the following steps:
a) selecting a protein comprising an ankyrin repeat domain with an N-terminal capping module that does not have L at position 17; and
b) replacing one or more amino acid residues of the protein to result in a protein of the invention comprising an ankyrin repeat domain with an N-terminal capping module having L at position 17.

As indicated above, a protein of the invention resulting from the replacement method comprises an ankyrin repeat domain with an N-terminal capping module having a mutation at position 17 of the N-terminal capping module, such as an amino acid residue selected from the group consisting of L, I and V, and may have one or more further mutations in the ankyrin repeat domain as specified herein. In some embodiments, the N-terminal capping module of the ankyrin repeat domain resulting from the replacement method comprises an amino acid sequence that has at least 70%, at least 75%, at least 80%, at least 85%, at least 90%, at least 95%, or 100% sequence identity to an amino acid sequence selected from the group consisting of SEQ ID NOs: 1 to 12. In some embodiments, the thermostability of the ankyrin repeat domain resulting from the replacement method is improved in comparison to a reference ankyrin repeat domain having the same amino acid sequence except for the amino acid residue at position 17 of the N-terminal capping module, which is, e.g., E in the reference ankyrin repeat domain. In some embodiments, the thermostability of the ankyrin repeat domain of the protein resulting from the replacement method is improved in comparison to the ankyrin repeat domain of the original protein.

The protein resulting from the replacement method can be further modified, mutated and/or optimized by methods commonly known in the art. In some embodiments, the protein resulting from the replacement method comprises one or more further moieties in addition to the ankyrin repeat domain as outlined above for the protein of the invention. Such modification with one or more further moieties may occur before, during or after the replacement of the one or more amino acid residues. In some embodiments, the one or more further moieties are added to the protein after replacement of the one or more amino acid residues. In some embodiments, the one or more further moieties are added to the protein before replacement of the one or more amino acid residues.

The present disclosure also relates to a method of designing or optimizing the amino acid sequence of an ankyrin repeat domain in silico through computational methods to result in a protein of the invention. It is to be understood that the ankyrin repeat domain may be entirely designed in silico or partially, e.g., by optimizing a pre-existing ankyrin repeat domain through computational methods.

Thus, in one embodiment, the present disclosure provides a method of designing a protein comprising designing or optimizing the amino acid sequence of an ankyrin repeat domain in silico through computational methods to result in a protein of the invention.

As indicated above, a protein of the invention resulting from such design method comprises an ankyrin repeat domain with an N-terminal capping module having a mutation at position 17 of the N-terminal capping module, such as an amino acid residue selected from the group consisting of L, I and V, and may have one or more further mutations as specified herein. In some embodiments, the N-terminal capping module of the in silico designed or optimized ankyrin repeat domain comprises an amino acid sequence that has at least 70%, at least 75%, at least 80%, at least 85%, at least 90%, at least 95%, or 100% sequence identity to an amino acid sequence selected from the group consisting of SEQ ID NOs: 1 to 12. In some embodiments, the thermostability of the designed or optimized ankyrin repeat domain is improved in comparison to a reference ankyrin repeat domain having the same amino acid sequence except for the amino acid residue at position 17 of the N-terminal capping module, which is, e.g., E in the reference ankyrin repeat domain.

The protein comprising the designed or optimized ankyrin repeat domain can be further modified, mutated and/or optimized by methods commonly known in the art. In some embodiments, the protein comprising the designed or optimized ankyrin repeat domain comprises one or more further moieties in addition to the ankyrin repeat domain as outlined above for the protein of the invention. Such modification with one or more further moieties may occur before, during or after the in silico design or optimization of the ankyrin repeat domain.

In some embodiments, the protein of the invention, e.g., a protein resulting from one of the above methods, is expressed or synthesized. In some embodiments, the expressed or synthesized protein is purified after its expression or synthesis. In some embodiments, the expressed or synthesized and, optionally, purified protein is formulated as a pharmaceutical composition.

In further aspects, the present disclosure provides a nucleic acid encoding the protein of the invention, a chromosome or vector comprising such nucleic acid, such as a bacterial vector, a viral vector or a synthetic vector (e.g., a LNP vector), and a cell or in vitro expression system comprising such nucleic acid, chromosome or vector.

The nucleic acid can be DNA or RNA, single-stranded or double-stranded, in isolated form or part of a larger nucleic acid, e.g., of a vector or a chromosome. The nucleic acid may comprise elements that enable delivery of the nucleic acid to a cell and/or expression of the nucleic acid in a cell. For instance, the nucleic acid encoding the protein of the invention can be operatively linked to expression control sequences, which have an impact on the transcription and/or translation of the protein, such as promoters, enhancers, transcription terminators, start codons and stop codons. Depending on the intended application and/or context, the expression control sequences may be selected from any eukaryotic or prokaryotic organism. Suitable promoters may be constitutive or inducible promoters. Examples include the CMV-, lacZ-, T7-, T5-, RSV-, SV40-, AOX1-, and GAPDH-promoter. Suitable enhancers include the CMV-enhancer, insulin-responsive elements, and SV40-enhancer. Suitable transcription terminators include the SV40-, lacZ-, and tk-polyadenylation signal.

The present disclosure also provides a library of nucleic acids comprising one or more nucleic acids encoding a protein of the invention. In some embodiments, the nucleic acid library comprises at least 10³, at least 10⁵, at least 10⁷, at least 10⁹, at least 10¹⁰, at least 10¹¹, at least 10¹² or at least 10¹³ nucleic acids encoding a protein comprising an ankyrin repeat domain, including one or more nucleic acids encoding a protein of the invention. In some embodiments, the nucleic acid library comprises at least 10³, at least 10⁵, at least 10⁷, at least 10⁹, at least 10¹⁰, at least 10¹¹, at least 10¹² or at least 10¹³ nucleic acids encoding a protein of the invention. In some embodiments, the nucleic acid library comprises at least 10³, at least 10⁵, at least 10⁷, at least 10⁹, at least 10¹⁰, at least 10¹¹, at least 10¹² or at least 10¹³ nucleic acids, each encoding a protein comprising an ankyrin repeat domain with a different amino acid sequence, and the library comprises one or more nucleic acids encoding a protein of the invention. In some embodiments, the nucleic acid library comprises at least 10³, at least 10⁵, at least 10⁷, at least 10⁹, at least 10¹⁰, at least 10¹¹, at least 10¹² or at least 10¹³ nucleic acids, each encoding a protein of the invention comprising an ankyrin repeat domain with a different amino acid sequence. In some embodiments, substantially all nucleic acids of the library encode a protein comprising an ankyrin repeat domain with a different amino acid sequence. In some embodiments, substantially all nucleic acids of the library encode a protein of the invention. In some embodiments, the nucleic acid library comprises at least one nucleic acid encoding a protein of the invention.

The cell comprising the nucleic acid, the chromosome or the vector of the invention can be a prokaryotic or a eukaryotic cell. In some embodiments, the cell is a bacterial, yeast or mammalian cell. In some embodiments, the cell is derived from E. coli, P. pastoris, S. cerevisiae, human, hamster or mouse. In some embodiments, the cell is selected from CHO, HEK293, BHK, NS0, Sp2/0, HT-1080, PER.C6, CAP and HuH-7 cells.

In some embodiments, the in vitro expression system comprising the nucleic acid, chromosome or vector of the invention is based on a cell-free extract from E. coli, yeast, rabbit, wheat germ, insect or human.

In a further aspect, the present disclosure provides a method of preparing a protein comprising the following steps:
a) culturing a cell comprising a nucleic acid encoding the protein of the invention under conditions allowing expression thereof; and
b) purifying the expressed protein.

In one embodiment, the present disclosure provides a method of preparing a protein comprising the following steps:
a) assembling by genetic means one or more gene(s) encoding the protein of the invention, and
b) expressing the gene(s) encoding the protein of the invention.

The present disclosure also provides a pharmaceutical composition comprising any one of the protein of the invention, the nucleic acid of the invention or the cell of the invention. In some embodiments, the pharmaceutical composition comprises an aqueous solution. For instance, it may comprise at least 1 wt % water. In some embodiments, the pharmaceutical composition comprises less than 1 wt % water. In some embodiments, the pharmaceutical composition is comprised in a glass or a plastic container.

In a further aspect, the present disclosure provides the use of any one of the protein of the invention, the nucleic acid of the invention or the cell of the invention in a method of treating a disease, condition or symptom. In a further aspect, the present disclosure provides a method of treating a disease, condition or symptom comprising the administration of any one of the protein of the invention, the nucleic acid of the invention or the cell of the invention. In a further aspect, the present disclosure provides the use of any one of the protein of the invention, the nucleic acid of the invention or the cell of the invention in the manufacture of a medicament for the treatment of a disease, condition or symptom. In some embodiments, the disease, condition or symptom is selected from the group consisting of cancer, an immunological disease, such as an autoimmune disease, a fibrotic disease, an inflammatory disease, an ophthalmological disease, a neurodegenerative disease, an infectious disease, a nephropathy, a cardiovascular disease and a metabolic disease.

Further embodiments are as follows:
E1. A protein comprising an ankyrin repeat domain,
   wherein the N-terminal capping module of the ankyrin repeat domain has mutation at position 17.
E2. The protein according to E1,
   wherein the mutation is an amino acid residue selected from the group consisting of L, I and V.
E3. The protein according to E1 or E2,
   wherein the N-terminal capping module comprises an amino acid sequence that has at least 70% sequence identity to an amino acid sequence selected from the group consisting of SEQ ID NOs: 1 to 60.
E4. The protein according to E1 or E2,
   wherein the N-terminal capping module comprises an amino acid sequence that has at least 70% sequence identity to an amino acid sequence selected from the group consisting of SEQ ID NOs: 1 to 12.
E5. A protein comprising an ankyrin repeat domain,
   wherein the N-terminal capping module of the ankyrin repeat domain (a) has an amino acid residue selected from the group consisting of L, I and V at position 17 and (b) comprises an amino acid sequence that has at least 70% sequence identity to an amino acid sequence selected from the group consisting of SEQ ID NOs: 1 to 60.
E6. A protein comprising an ankyrin repeat domain,
   wherein the N-terminal capping module (a) has an amino acid residue selected from L and I at position 17 and (b) comprises an amino acid sequence that has at least 70% sequence identity to an amino acid sequence selected from the group consisting of SEQ ID NOs: 1 to 60.
E7. The protein according to any one of E1 to E6,
   wherein the ankyrin repeat domain has a higher melting temperature than a reference ankyrin repeat domain having the same amino acid sequence except for the amino acid residue at position 17 of the N-terminal capping module, which is E in the reference ankyrin repeat domain.
E8. The protein according to any one of E1 to E7,
   wherein the N-terminal capping module of the ankyrin repeat domain further has an amino acid residue selected from the group consisting of I, T, A, V, L and M at position 15.
E9. The protein according to any one of E1 to E8,
   wherein the N-terminal capping module of the ankyrin repeat domain further has I at position 15.
E10. The protein according to any one of E1 to E9,
   wherein the internal ankyrin repeat of the ankyrin repeat domain that is adjacent to the N-terminal capping module has L or I at position 23.
E11. The protein according to E9,
   wherein the internal ankyrin repeat of the ankyrin repeat domain that is adjacent to the N-terminal capping module has L at position 23.
E12. The protein according to any one of E1 to E11,
   wherein the N-terminal capping module of the ankyrin repeat domain has an amino acid residue selected from the group consisting of L, V and I at position 22.
E13. The protein according to any one of E1 to E12,
   wherein the N-terminal capping module of the ankyrin repeat domain has L at position 24.
E14. The protein according to any one of E1 to E13, wherein said protein comprises one or more further ankyrin repeat domains.
E15. A nucleic acid comprising a sequence encoding a protein according to any one of E1 to E14.
E16. A vector or cell comprising the nucleic acid according to E15.
E17. A library of proteins comprising one or more proteins according to any one of E1 to E14.
E18. A method for selecting a protein that specifically binds to a target comprising the following steps:
   (i) providing the library of proteins according to E17; and
   (ii) selecting a protein specifically binding to the target via its ankyrin repeat domain from the library, wherein the selected protein is a protein according to any one of E1 to E14.
E19. A method of preparing a protein comprising the following steps (A) or (B):
   (A)(i) selecting a protein comprising an ankyrin repeat domain with an N-terminal capping module that does not have an amino acid residue selected from the group consisting of L, I and V at position 17; and
      (ii) replacing one or more amino acid residues of the protein to result in a protein according to any one of E1 to E14; or
   (B)(i) selecting a protein comprising an ankyrin repeat domain with an N-terminal capping module that does not have an amino acid residue selected from L or I at position 17; and
      (ii) replacing one or more amino acid residues of the protein to result in a protein according to any one of E1 to E14 having L or I at position 17 of the N-terminal capping module of the ankyrin repeat domain.
E20. A method of preparing a protein comprising designing or optimizing the amino acid sequence of an ankyrin repeat domain in silico through computational methods to result in a protein according to any one of E1 to E14.
E21. The method according to any one of E18 to E20, wherein the resulting protein is further modified with one or more further ankyrin repeat domains.
E22. A method of producing a protein comprising the following steps:
   (i) expressing or synthesizing a protein according to any one of E1 to E14 or a protein resulting from the method according to any one of E18 to E21; and
   (ii) purifying the expressed or synthesized protein.
E23. The method according to E22, further comprising the following step:
   (iii) formulating the purified protein as a pharmaceutical composition.
E24. A protein resulting from the method according to any one of E18 to E22.
E25.A pharmaceutical composition comprising any one of the following: the protein according to any one of E1 to E14 and E24, the nucleic acid according to E15 and the vector or cell according to E16,
wherein the pharmaceutical composition further comprises a pharmaceutically acceptable carrier.

### EXAMPLES

### Materials and Methods

### Protein Sequences

His-tagged ankyrin repeat domains P #99 to P #132 corresponding to SEQ ID NOs: 99 to 132, respectively, were tested.

The DNA sequence encoding each ankyrin repeat domain was chemically synthesized and cloned into pQIq expression vectors (Simon M. et al., Bioconjug Chem., 23 (2), 279-86, 2012) by standard techniques.

### Protein Expression

The ankyrin repeat domains were expressed in E. coli BL21 or XL1-Blue cells and purified via their His-tag using standard protocols. Briefly, 25 ml of stationary overnight cultures (LB, 1% glucose, 100 mg/l of ampicillin; 37° C.) were used to inoculate 1 I cultures (same medium). At an absorbance of about 1 at 600 nm, the cultures were induced with 0.5 mM IPTG and incubated at 37° C. for 4 h. The cultures were centrifuged and the resulting pellets were resuspended in 40 ml of TBS500 (50 mM Tris-HCl, 500 mM NaCl, pH 8) and sonicated. The lysate was recentrifuged, and glycerol (10% (v/v) final concentration) and imidazole (20 mM final concentration) were added to the resulting supernatant. The ankyrin repeat domains were purified over a Ni-nitrilotriacetic acid column (2.5 ml column volume) according to the manufacturer's instructions (QIAgen, Germany). Up to 200 mg of highly soluble ankyrin repeat domains were purified from one liter of E. coli culture with a purity >95% as estimated from SDS-15% PAGE. Such purified ankyrin repeat domains were used for further characterizations.

### CD Measurement

The CD signal of the ankyrin repeat domains was recorded at 222 nm in a Chirascan V100 instrument (Applied Photophysics). To this end, each sample was slowly heated from 25° C. to 100° C. using a temperature ramp of 1° C. per min and collecting data periodically at 0.8° C. intervals. Each sample contained an ankyrin repeat domain at a concentration of 0.01 mM in a HEPES buffer (10 mM Hepes, 150 mM NaCl, pH 7.4). Depending on the sample and, as indicated below, 1 M, 2 M, 4 M or 6 M guanidine hydrochloride (GdmCl) was additionally added to the HEPES buffer to allow for proper unfolding of the ankyrin repeat domain.

Measuring the CD signal of ankyrin repeat domains is an effective means to follow their denaturation as they mainly consist of alpha helices that show a strong change in their CD signal at 222 nm upon unfolding. The midpoint of the observed transition of such a measured CD signal trace for an ankyrin repeat domain corresponds to its Tm value. Tm values were derived as described in V. Consalvi et al. (Protein Eng Des Sel. 13, 501-507, 2000).

### Example 1

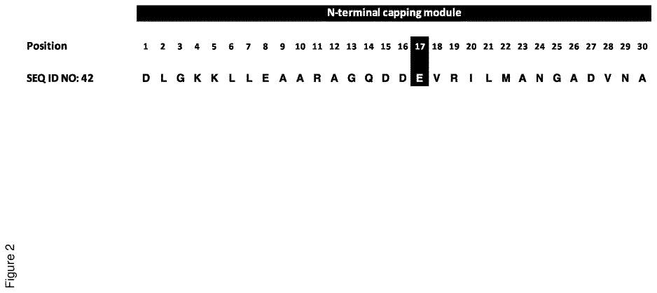

The archetypal N-terminal capping module of designed ankyrin repeat domains (Binz, 2003, loc. cit.), which is still commonly used today in scientific studies, comprises an E at position 17 (see SEQ ID NO: 42 and FIG. 2). Based on the results of in silico studies, the present inventors mutated this position 17 into various other amino acid residues.

Table 2 below shows the melting temperatures of a set of ankyrin repeat domains, P #99-P #103 (corresponding to SEQ ID NOs: 99-103), which only differ from each other in the amino acid residue at position 17 of their N-terminal capping module (the sample buffer contained no GdmCl):

TABLE 2

| Protein | Position 17 | Tm value [°C] |
|---|---|---|
| P#99 | E | 71.4 |
| P#100 | A | 71.7 |
| P#101 | Q | 71.5 |
| P#102 | K | 71.7 |
| P#103 | I | 73.3 |

As reflected in Table 2 above, the mutation I at position 17 of the N-terminal capping module showed the largest increase in thermostability (the N-terminal capping module of the corresponding protein finds reflection in SEQ ID NO: 1).

### Example 2

This mutation I at position 17 of the N-terminal capping module was tested, as well as yet other mutations at position 17 of the N-terminal capping module, in an ankyrin repeat domain of a different sequence background, including a much different N-terminal capping module. The corresponding ankyrin repeat domains P #104-P #108 (corresponding to SEQ ID NOs: 104-108) only differ from each other in the amino acid residue at position 17 of the N-terminal capping module. The melting curves of these molecules were determined in sample buffer containing 4 M GdmCl and the corresponding melting temperatures are shown in Table 3:

TABLE 3

| Protein | Position 17 | Tm value [°C] |
|---|---|---|
| P#104 | E | 72.2 |
| P#105 | I | 73.6 |
| P#106 | V | 73.2 |
| P#107 | L | 75.4 |
| P#108 | T | 70.7 |

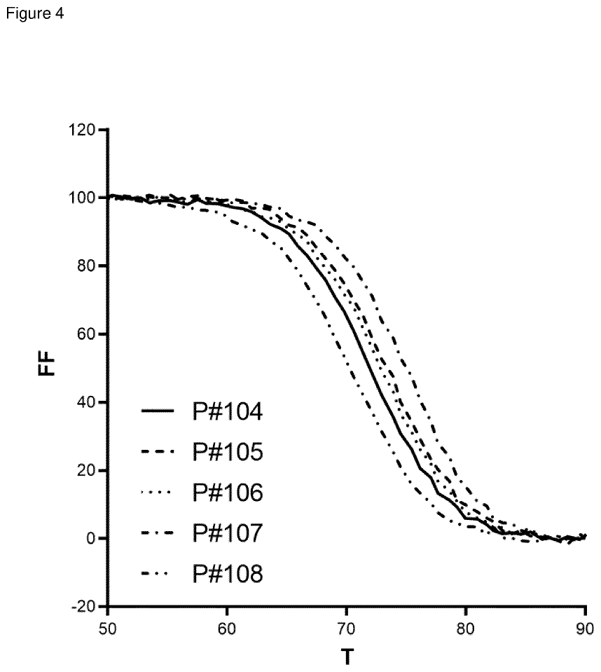

The corresponding melting curves of P #104-P #108 are shown in FIG. 4.

### Example 3

As shown in Table 3, the mutations I, V and L at position 17 of the N-terminal capping module increased thermostability as compared to E at this position and were thus further tested in yet another sequence background (the N-terminal capping module of P #107 also finds reflection in SEQ ID NO: 2). The corresponding ankyrin repeat domains P #109-P #112 (corresponding to SEQ ID NOs: 109-112) once again only differ from each other in the amino acid residue at position 17 of the N-terminal capping module. The melting curves of these molecules were determined in sample buffer containing 2 M GdmCl and the corresponding melting temperatures are shown in Table 4:

TABLE 4

| Protein | Position 17 | Tm value [°C] |
|---|---|---|
| P#109 | E | 75.4 |
| P#110 | L | 78.4 |
| P#111 | I | 77.6 |
| P#112 | V | 77.4 |

### Example 4

As shown in Table 4, the three mutations I, V and L at position 17 of the N-terminal capping module were confirmed to also increase thermostability in this further sequence background (the N-terminal capping module of P #110 also finds reflection in SEQ ID NO: 3). The mutation L at this position was tested in five further sets of sequence backgrounds, each of which only differs in the amino acid residue at position 17 of the N-terminal capping module.

The melting curves of the first set, P #113 and P #114 (corresponding to SEQ ID NOs: 113 and 114), were determined in sample buffer containing 1 M GdmCl and the corresponding melting temperatures are shown in Table 5 (the N-terminal capping module of P #114 also finds reflection in SEQ ID NO: 4):

TABLE 5

| Protein | Position 17 | Tm value [°C] |
|---|---|---|
| P#113 | E | 52.6 |
| P#114 | L | 58.4 |

The melting curves of the second set, P #115 and P #116 (corresponding to SEQ ID NOs: 115 and 116), were determined in sample buffer containing 2 M GdmCl and the corresponding melting temperatures are shown in Table 6 (the N-terminal capping module of P #116 also finds reflection in SEQ ID NO: 5):

TABLE 6

| Protein | Position 17 | Tm value [°C] |
|---|---|---|
| P#115 | A | 63.9 |
| P#116 | L | 68.8 |

The melting curves of the third set, P #117 and P #118 (corresponding to SEQ ID NOs: 117 and 118), were determined in sample buffer containing 6 M GdmCl and the corresponding melting temperatures are shown in Table 7 (the N-terminal capping module of P #118 also finds reflection in SEQ ID NO: 6):

TABLE 7

| Protein | Position 17 | Tm value [°C] |
|---|---|---|
| P#117 | E | 61.2 |
| P#118 | L | 64.4 |

The melting curves of the fourth set, P #119 and P #120 (corresponding to SEQ ID NOs: 119 and 120), were determined in sample buffer containing 4 M GdmCl and the corresponding melting temperatures are shown in Table 8 (the N-terminal capping module of P #120 also finds reflection in SEQ ID NO: 7):

TABLE 8

| Protein | Position 17 | Tm value [°C] |
|---|---|---|
| P#119 | E | 77.4 |
| P#120 | L | 80.3 |

The melting curves of the fifth set, P #121 and P #122 (corresponding to SEQ ID NOs: 121 and 122), were determined in sample buffer containing 4 M GdmCl and the corresponding melting temperatures are shown in Table 9 (the N-terminal capping module of P #122 also finds reflection in SEQ ID NO: 8):

TABLE 9

| Protein | Position 17 | Tm value [°C] |
|---|---|---|
| P#121 | E | 78.4 |
| P#122 | L | 81.0 |

### Example 5

To further confirm the broad applicability of the mutation at position 17 of the N-terminal capping module, it was tested in five further sets of ankyrin repeat domains with diverging sequences, each set only differing in the amino acid residue at position 17 of the N-terminal capping module of the ankyrin repeat domain.

The melting curves of the first set, P #123 and P #124 (corresponding to SEQ ID NOs: 123 and 124), were determined in sample buffer containing 4 M GdmCl and the corresponding melting temperatures are shown in Table 10 (the N-terminal capping module of P #124 also finds reflection in SEQ ID NO: 9):

TABLE 10

| Protein | Position 17 | Tm value [°C] |
|---|---|---|
| P#123 | E | 74.9 |
| P#124 | L | 79.0 |

The melting curves of the second set, P #125 and P #126 (corresponding to SEQ ID NOs: 125 and 126), were determined in sample buffer containing 2 M GdmCl and the corresponding melting temperatures are shown in Table 11 (the N-terminal capping module of P #126 also finds reflection in SEQ ID NO: 9):

TABLE 11

| Protein | Position 17 | Tm value [°C] |
|---|---|---|
| P#125 | E | 66.4 |
| P#126 | L | 70.3 |

The melting curves of the third set, P #127 and P #128 (corresponding to SEQ ID NOs: 127 and 128), were determined in sample buffer without GdmCl and the corresponding melting temperatures are shown in Table 12 (the N-terminal capping module of P #128 also finds reflection in SEQ ID NO: 10):

TABLE 12

| Protein | Position 17 | Tm value [°C] |
|---|---|---|
| P#127 | E | 71.6 |
| P#128 | L | 76.6 |

The melting curves of the fourth set, P #129 and P #130 (corresponding to SEQ ID NOs: 129 and 130), were determined in sample buffer containing 2 M GdmCl and the corresponding melting temperatures are shown in Table 13 (the N-terminal capping module of P #130 also finds reflection in SEQ ID NO: 11):

TABLE 13

| Protein | Position 17 | Tm value [°C] |
|---|---|---|
| P#129 | E | 62.5 |
| P#130 | L | 66.4 |

The melting curves of the fifth set, P #131 and P #132 (corresponding to SEQ ID NOs: 131 and 132), were determined in sample buffer containing 4 M GdmCl and the corresponding melting temperatures are shown in Table 14 (the N-terminal capping module of P #132 also finds reflection in SEQ ID NO: 12):

TABLE 14

| Protein | Position 17 | Tm value [°C] |
|---|---|---|
| P#131 | E | 82.6 |
| P#132 | L | 84.7 |

As demonstrated above, various mutations at position 17 of the N-terminal capping module cause an increase in thermostability of the ankyrin repeat domain in diverse sequence backgrounds, suggesting that the stabilizing effect of these mutations is broadly applicable. In particular, the mutations L, I and V showed a consistent and marked increase in thermostability of up to 5.8° C.

1. A protein comprising an ankyrin repeat domain, wherein the N-terminal capping module of the ankyrin repeat domain (A) comprises an amino acid sequence that has at least 70% sequence identity to an amino acid sequence selected from the group consisting of SEQ ID NOS: 1 to 12 and (B) has an amino acid residue selected from the group consisting of L, I and V at the position corresponding to position 17 of SEQ ID NO: 1.

2. The protein according to claim 1, wherein said ankyrin repeat domain has a higher melting temperature than a reference ankyrin repeat domain having the same amino acid sequence except for the amino acid residue at the position corresponding to position 17 of SEQ ID NO: 1, which is E in the reference ankyrin repeat domain.

3. The protein according to claim 1, wherein the N-terminal capping module of the ankyrin repeat domain comprises an amino acid sequence that has at least 75%, at least 80%, at least 85%, at least 90%, at least 95% or 100% sequence identity to an amino acid sequence selected from the group consisting of SEQ ID NOS:
to 12.

4. The protein according to claim 1, wherein the amino acid residue at the position corresponding to position 17 of SEQ ID NO: 1 is selected from L or I.

5. The protein according to claim 1, wherein the N-terminal capping module of the ankyrin repeat domain comprises an amino acid sequence selected from the group consisting of SEQ ID NOs: 1 to 60.

6. A nucleic acid comprising a sequence encoding a protein according to claim 1.

7. A vector or cell comprising the nucleic acid according to claim 6.

8. A library of proteins comprising one or more proteins according to claim 1.

9. A method for selecting a protein that specifically binds to a target comprising the following steps:
(i) providing the library of proteins according to claim 8; and
(ii) selecting a protein from the library that specifically binds to the target via said ankyrin repeat domain.

10. A method of preparing a protein comprising:
(i) selecting a protein comprising an ankyrin repeat domain with an N-terminal capping module that does not have an amino acid residue selected from the group consisting of L, I and V at the position corresponding to position 17 of SEQ ID NO: 1; and
(ii) replacing one or more amino acid residues of the protein to result in a protein according to claim 1; or
designing or optimizing the amino acid sequence of an ankyrin repeat domain in silico through computational methods to result in a protein according to claim 1.

11. The method according to claim 9, wherein one or more further ankyrin repeat domains become bound to the resulting protein.

12. A method of producing a protein comprising the following steps:
(i) expressing or synthesizing a protein according to claim 1; and
(ii) purifying the expressed or synthesized protein.

13. The method according to claim 12, further comprising the following step:
(iii) formulating the purified protein as a pharmaceutical composition.

14. A protein resulting from the method according to claim 9.

15. A pharmaceutical composition comprising one or more of the following: the protein according to claim 1 or a nucleic acid comprising a sequence encoding the protein according to claim 1 or a vector comprising the nucleic acid,
wherein the pharmaceutical composition further comprises a pharmaceutically acceptable carrier.

16. The method according to claim 10, wherein one or more further ankyrin repeat domains become bound to the resulting protein.

17. A method of preparing a protein comprising:
(i) selecting a protein comprising an ankyrin repeat domain with an N-terminal capping module that does not have an amino acid residue selected from L or I at the position corresponding to position 17 of SEQ ID NO: 1; and
(ii) replacing one or more amino acid residues of the protein to result in a protein according to claim 4.

18. The method according to claim 17, wherein one or more further ankyrin repeat domains become bound to the resulting protein.

19. A method of producing a protein comprising the following steps:
(i) expressing or synthesizing a protein resulting from the method according to claim 9; and
(ii) purifying the expressed or synthesized protein.

20. A method of producing a protein comprising the following steps:
(i) expressing or synthesizing a protein resulting from the method according to claim 17; and
(ii) purifying the expressed or synthesized protein.


SEQUENCE LISTING (132 sequences)

SEQ ID NO: 1 - protein, 30 aa, synthetic construct
DLAKKLLEAARAGQDDIVRELLKAGADVNA

SEQ ID NO: 2 - protein, 30 aa, synthetic construct
DLGKKLLEAAAAGQIDLVKKLIALGADVNA

SEQ ID NO: 3 - protein, 30 aa, synthetic construct
DLGKKLLEAARAGQDDLVRELLKAGADVNA

SEQ ID NO: 4 - protein, 30 aa, synthetic construct
DLDKKLLEAARAGQDDLVRILMANGADVNA

SEQ ID NO: 5 - protein, 30 aa, synthetic construct
DLAKKLLAAAAAGQLELVEQLLKAGADVNA

SEQ ID NO: 6 - protein, 30 aa, synthetic construct
DLGKKLLQAARAGQLDLVRELLKAGADVNA

SEQ ID NO: 7 - protein, 30 aa, synthetic construct
DLGKKLLEAARAGQIDLVKELIALGADVNA

SEQ ID NO: 8 - protein, 30 aa, synthetic construct
DLGKKLLEAARAGQIDLVKKLIALGADVNA

SEQ ID NO: 9 - protein, 30 aa, synthetic construct
DLGKKLLEAAAAGQIDLVKELIALGADVNA

SEQ ID NO: 10 - protein, 30 aa, synthetic construct
DLDKKLLEAARAGQDDLVRILLKAGADVNA

SEQ ID NO: 11 - protein, 30 aa, synthetic construct
DLDKKLLEAAAAGQIDLVKELIALGADVNA

SEQ ID NO: 12 - protein, 30 aa, synthetic construct
DLGKKLLQAAAAGQIDLVKELIALGADVNA

SEQ ID NO: 13 - protein, 30 aa, synthetic construct
DLGKKLLEAAAAGQLDLVRILIALGADVNA

SEQ ID NO: 14 - protein, 30 aa, synthetic construct
DLGKKLLEAAAAGQLDVVKELIALGADVNA

SEQ ID NO: 15 - protein, 30 aa, synthetic construct
DLGKKLLEAARAGQLDIVRELLKLGADVNA

SEQ ID NO: 16 - protein, 30 aa, synthetic construct
DLGKKLLEAAEAGQVDLVKELLKLGADVNA

SEQ ID NO: 17 - protein, 30 aa, synthetic construct
DLGKKLLEAAEAGQVDIVKILLKLGADVNA

SEQ ID NO: 18 - protein, 30 aa, synthetic construct
DLGKKLLEAAEAGQIDLVKELIALGADVNA

SEQ ID NO: 19 - protein, 30 aa, synthetic construct
DLGKKLLEAAAAGQVDLVRELIKLGADVNA

SEQ ID NO: 20 - protein, 30 aa, synthetic construct
DLGKKLLEAAAAGQVDVVKELIKLGADVNA

SEQ ID NO: 21 - protein, 30 aa, synthetic construct
DLGKKLLEAARAGQLDIVRILIKLGADVNA

SEQ ID NO: 22 - protein, 30 aa, synthetic construct
DLGKKLLEAARAGQLDLVKILIKLGADVNA

SEQ ID NO: 23 - protein, 30 aa, synthetic construct
DLGKKLLEAAEAGQLDVVRELLALGADVNA

SEQ ID NO: 24 - protein, 30 aa, synthetic construct
DLGKKLLEAAEAGQLDIVKELLALGADVNA

SEQ ID NO: 25 - protein, 30 aa, synthetic construct
DLGKKLLEAAAAGQVDLVKELIALGADVNA

SEQ ID NO: 26 - protein, 30 aa, synthetic construct
DLDKKLLEAAEAGQIDLVKELIALGADVNA

SEQ ID NO: 27 - protein, 30 aa, synthetic construct
DLGKKLLEAARAGQIDVVKILLALGADVNA

SEQ ID NO: 28 - protein, 30 aa, synthetic construct
DLAKKLLEAAAAGQIDLVKELIALGADVNA

SEQ ID NO: 29 - protein, 30 aa, synthetic construct
DLGKKLLEAAAAGQIDIVRELIALGADVNA

SEQ ID NO: 30 - protein, 30 aa, synthetic construct
DLGKKLLEAAEAGQIDVVRILIALGADVNA

SEQ ID NO: 31 - protein, 30 aa, synthetic construct
DLGKKLLEAAEAGQIDIVKILIALGADVNA

SEQ ID NO: 32 - protein, 30 aa, synthetic construct
DLAKKLLEAAEAGQIDLVKELIALGADVNA

SEQ ID NO: 33 - protein, 30 aa, synthetic construct
DLGKKLLEAARAGQIDEVRELLKAGADVNA

SEQ ID NO: 34 - protein, 30 aa, synthetic construct
DLDKKLLAAAEAGQIDLVKELIALGADVNA

SEQ ID NO: 35 - protein, 30 aa, synthetic construct
DLGKKLLEAARAGQVDEVRELIKAGADVNA

SEQ ID NO: 36 - protein, 30 aa, synthetic construct
DLGKKLLAAAAAGQVEAVEQLIKAGADVNA

SEQ ID NO: 37 - protein, 30 aa, synthetic construct
DLGKKLLEAARAGQLDEVRELLKAGADVNA

SEQ ID NO: 38 - protein, 30 aa, synthetic construct
DLGKKLLEAAEAGQVDEVKILLKAGADVNA

SEQ ID NO: 39 - protein, 30 aa, synthetic construct
DLGKKLLEAAEAGQLDEVKELLAAGADVNA

SEQ ID NO: 40 - protein, 30 aa, synthetic construct
DLGKKLLEAAAAGQIEIVKKLIALGADVNA

SEQ ID NO: 41 - protein, 30 aa, synthetic construct
DLGKKLLQAAAAGQIQIVKKLIALGADVNA

SEQ ID NO: 42 - protein, 30 aa, synthetic construct
DLGKKLLEAARAGQDDEVRILMANGADVNA

SEQ ID NO: 43 - protein, 30 aa, synthetic construct
DLGKKLLEAARAGQDDEVRELLKAGADVNA

SEQ ID NO: 44 - protein, 30 aa, synthetic construct
DLGKKLLEAARAGQDDEVRILLKAGADVNA

SEQ ID NO: 45 - protein, 30 aa, synthetic construct
DLDKKLLEAARAGQDDEVRILMANGADVNA

SEQ ID NO: 46 - protein, 30 aa, synthetic construct
DLGKKLLEAARAGQDDEVRILIANGAPFTT

SEQ ID NO: 47 - protein, 30 aa, synthetic construct
DLGKKLLEAARAGQDDEVRELTKAGADVNA

SEQ ID NO: 48 - protein, 30 aa, synthetic construct
DLGKKLLEAARAGQDDEVHILIANGADVNA

SEQ ID NO: 49 - protein, 30 aa, synthetic construct
DLGKKLLQAARAGQLDEVRELLKAGADVNA

SEQ ID NO: 50 - protein, 30 aa, synthetic construct
DLGKKLLKAARAGQKDEVKILMENGADVNA

SEQ ID NO: 51 - protein, 30 aa, synthetic construct
DLGKKLLEAARAGQDDEVRILMALGADVNA

SEQ ID NO: 52 - protein, 30 aa, synthetic construct
DLGKKLLEAARAGQIDLVRELLKLGADVNA

SEQ ID NO: 53 - protein, 30 aa, synthetic construct
DLDKKLLEAARAGQLDVVRELTKLGADVNA

SEQ ID NO: 54 - protein, 30 aa, synthetic construct
DLGKKLLEAARAGQVDIVRELIKLGADVNA

SEQ ID NO: 55 - protein, 30 aa, synthetic construct
DLDKKLLEAAAAGQIDLVRELLKLGADVNA

SEQ ID NO: 56 - protein, 30 aa, synthetic construct
DLAKKLLEAAAAGQLDVVRELVKLGADVNA

SEQ ID NO: 57 - protein, 30 aa, synthetic construct
DLGKKLLEAAAAGQVDIVRELIKLGADVNA

SEQ ID NO: 58 - protein, 30 aa, synthetic construct
DLAKKLLEAAAAGQIDLVRELAKLGADVNA

SEQ ID NO: 59 - protein, 30 aa, synthetic construct
DLAKKLLAAAAAGQLEVVEQLLKLGADVNA

SEQ ID NO: 60 - protein, 30 aa, synthetic construct
DLAKKLLAAAAAGQLEAVEQLLKAGADVNA

SEQ ID NO: 61 - protein, 33 aa, synthetic construct
XDXXGXTPLHLAAXXGHLEIVEVLLKXGADVNA

SEQ ID NO: 62 - protein, 33 aa, synthetic construct
XDXXGXTPLHLAXXXGXXXVVXLLLXXGADVNA

SEQ ID NO: 63 - protein, 33 aa, synthetic construct
SXXXGXTPLHXAAXXGHLEIVDILLAXGADVNA

SEQ ID NO: 64 - protein, 33 aa, synthetic construct
XDXXGXTPLHLAAXXGHLEIVEILLKNGADVNA

SEQ ID NO: 65 - protein, 33 aa, synthetic construct
KDXXGXTPLHLAAXXGHLEIVELLLKAGADVNA

SEQ ID NO: 66 - protein, 33 aa, synthetic construct
XDXXGXTPLHXAAXXGHLEVVKLLLEXGADVNA

SEQ ID NO: 67 - protein, 33 aa, synthetic construct
XDXXGXTPLHLAAXXGHLEIVKLLLEAGADVNA

SEQ ID NO: 68 - protein, 33 aa, synthetic construct
XDXXGXTPLHLAAXXGHIEIVELLLKAGADVNA

SEQ ID NO: 69 - protein, 33 aa, synthetic construct
KDXXGXTPLHLAAXXGHIEIVKLLLEAGADVNA

SEQ ID NO: 70 - protein, 33 aa, synthetic construct
XDXXGXTPLHLAAXXGHLEIVELLLKXGADVNA

SEQ ID NO: 71 - protein, 33 aa, synthetic construct
SXXXGXTPLHXAAXXGHLEIVDLLLAXGADVNA

SEQ ID NO: 72 - protein, 33 aa, synthetic construct
XDXXGXTPLHLAAXXGHLEIVEVLLKNGADVNA

SEQ ID NO: 73 - protein, 33 aa, synthetic construct
KDXXGXTPLHLAAXXGHLEIVEVLLKAGADVNA

SEQ ID NO: 74 - protein, 33 aa, synthetic construct
KDXXGXTPLHXAAXXGHIEIVKLLLEAGADVNA

SEQ ID NO: 75 - protein, 33 aa, synthetic construct
EDXXGXTPLHLAAXXGHIEIVKLLLEAGADVNA

SEQ ID NO: 76 - protein, 33 aa, synthetic construct
SDXXGXTPLHLAAXXGHIEIVKLLLEAGADVNA

SEQ ID NO: 77 - protein, 28 aa, synthetic construct
QDKFGKTAFDISIDNGNEDLAEILQKLN

SEQ ID NO: 78 - protein, 28 aa, synthetic construct
QDKFGKTAFDISIDNGNEDLAEILQKAA

SEQ ID NO: 79 - protein, 28 aa, synthetic construct
QDKFGKTPFDLAIDNGNEDIAEVLQKAA

SEQ ID NO: 80 - protein, 28 aa, synthetic construct
QDKFGKTPFDLAIREGHEDIAEVLQKAA

SEQ ID NO: 81 - protein, 28 aa, synthetic construct
QDKSGKTPADLAADAGHEDIAEVLQKAA

SEQ ID NO: 82 - protein, 28 aa, synthetic construct
QDSAGKTPADLAADAGHEEIAEVLQKAA

SEQ ID NO: 83 - protein, 28 aa, synthetic construct
QDKAGLTPADLAADAGHEEIAEVLQKAA

SEQ ID NO: 84 - protein, 28 aa, synthetic construct
QDSLGKTPADLAALAGHEEIAEVLQKAA

SEQ ID NO: 85 - protein, 28 aa, synthetic construct
QDKAGQTPADLAADAGHEEIAEVLQKAA

SEQ ID NO: 86 - protein, 28 aa, synthetic construct
QDXXGXTPADLAAXAGHEEIAEVLQKAA

SEQ ID NO: 87 - protein, 28 aa, synthetic construct
QDKSGKTPADLAARAGHQDIAEVLQKAA

SEQ ID NO: 88 - protein, 28 aa, synthetic construct
QDKFGKTAEDLAKDNGNQDIADVLEKAL

SEQ ID NO: 89 - protein, 27 aa, synthetic construct
QDKFGKTAFDISIDNGNEELAKVLQDH

SEQ ID NO: 90 - protein, 28 aa, synthetic construct
QDKSGKTPADLAADAGHEDIVKLLQEAA

SEQ ID NO: 91 - protein, 28 aa, synthetic construct
QDKFGKTAEDLAKDNGNQDIADLLEKAL

SEQ ID NO: 92 - protein, 28 aa, synthetic construct
QDKSGKTPADLAADAGHEDIAKVLQEAA

SEQ ID NO: 93 - protein, 28 aa, synthetic construct
QDKSGKTPADLAADAGHEDIVEVLQKAA

SEQ ID NO: 94 - protein, 28 aa, synthetic construct
QDKSGKTPADLAADAGHEDIAELLQKAA

SEQ ID NO: 95 - protein, 28 aa, synthetic construct
QDKSGKTPADLAADAGHEDIVELLQKAA

SEQ ID NO: 96 - protein, 24 aa, synthetic construct
GSPTPTPTTPTPTPTTPTPTPTGS

SEQ ID NO: 97 - protein, 22 aa, synthetic construct
GSPTPTPTTPTPTPTTPTPTPT

SEQ ID NO: 98 - protein, 5 aa, synthetic construct
GGGGS

SEQ ID NO: 99 - protein, 103 aa, synthetic construct
MRGSHHHHHHGSDLAKKLLEAARAGQDDEVRELLKAGADVNAKDKDGYTPLHLAAREGHLEIVEVLLKAGADVNAQDKFGKTAFDISIDNGNEDLAEILQKLN

SEQ ID NO: 100 - protein, 103 aa, synthetic construct
MRGSHHHHHHGSDLAKKLLEAARAGQDDAVRELLKAGADVNAKDKDGYTPLHLAAREGHLEIVEVLLKAGADVNAQDKFGKTAFDISIDNGNEDLAEILQKLN

SEQ ID NO: 101 - protein, 103 aa, synthetic construct
MRGSHHHHHHGSDLAKKLLEAARAGQDDQVRELLKAGADVNAKDKDGYTPLHLAAREGHLEIVEVLLKAGADVNAQDKFGKTAFDISIDNGNEDLAEILQKLN

SEQ ID NO: 102 - protein, 103 aa, synthetic construct
MRGSHHHHHHGSDLAKKLLEAARAGQDDKVRELLKAGADVNAKDKDGYTPLHLAAREGHLEIVEVLLKAGADVNAQDKFGKTAFDISIDNGNEDLAEILQKLN

SEQ ID NO: 103 - protein, 103 aa, synthetic construct
MRGSHHHHHHGSDLAKKLLEAARAGQDDIVRELLKAGADVNAKDKDGYTPLHLAAREGHLEIVEVLLKAGADVNAQDKFGKTAFDISIDNGNEDLAEILQKLN

SEQ ID NO: 104 - protein, 103 aa, synthetic construct
MRGSHHHHHHGSDLGKKLLEAAAAGQIDEVKKLIALGADVNAKDKDGYTPLHLAAREGHLEIVKLLLEAGADVNAQDKSGKTPADLAADAGHEKIAELLQKAA

SEQ ID NO: 105 - protein, 103 aa, synthetic construct
MRGSHHHHHHGSDLGKKLLEAAAAGQIDIVKKLIALGADVNAKDKDGYTPLHLAAREGHLEIVKLLLEAGADVNAQDKSGKTPADLAADAGHEKIAELLQKAA

SEQ ID NO: 106 - protein, 103 aa, synthetic construct
MRGSHHHHHHGSDLGKKLLEAAAAGQIDVVKKLIALGADVNAKDKDGYTPLHLAAREGHLEIVKLLLEAGADVNAQDKSGKTPADLAADAGHEKIAELLQKAA

SEQ ID NO: 107 - protein, 103 aa, synthetic construct
MRGSHHHHHHGSDLGKKLLEAAAAGQIDLVKKLIALGADVNAKDKDGYTPLHLAAREGHLEIVKLLLEAGADVNAQDKSGKTPADLAADAGHEKIAELLQKAA

SEQ ID NO: 108 - protein, 103 aa, synthetic construct
MRGSHHHHHHGSDLGKKLLEAAAAGQIDTVKKLIALGADVNAKDKDGYTPLHLAAREGHLEIVKLLLEAGADVNAQDKSGKTPADLAADAGHEKIAELLQKAA

SEQ ID NO: 109 - protein, 136 aa, synthetic construct
MRGSHHHHHHGSDLGKKLLEAARAGQDDEVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAA

SEQ ID NO: 110 - protein, 136 aa, synthetic construct
MRGSHHHHHHGSDLGKKLLEAARAGQDDLVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAA

SEQ ID NO: 111 - protein, 136 aa, synthetic construct
MRGSHHHHHHGSDLGKKLLEAARAGQDDIVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAA

SEQ ID NO: 112 - protein, 136 aa, synthetic construct
MRGSHHHHHHGSDLGKKLLEAARAGQDDVVRELLKAGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAA

SEQ ID NO: 113 - protein, 136 aa, synthetic construct
MRGSHHHHHHGSDLDKKLLEAARAGQDDEVRILMANGADVNARDSTGWTPLHLAAPWGHPEIVEVLLKNGADVNAADFQGWTPLHLAAAVGHLEIVEVLLKYGADVNAQDKFGKTAFDISIDNGNEDLAEILQKAA

SEQ ID NO: 114 - protein, 136 aa, synthetic construct
MRGSHHHHHHGSDLDKKLLEAARAGQDDLVRILMANGADVNARDSTGWTPLHLAAPWGHPEIVEVLLKNGADVNAADFQGWTPLHLAAAVGHLEIVEVLLKYGADVNAQDKFGKTAFDISIDNGNEDLAEILQKAA

SEQ ID NO: 115 - protein, 103 aa, synthetic construct
MRGSHHHHHHDADLAKKLLAAAAAGQLEAVEQLLKAGADVNAKDKDGYTPLHLAAREGHLEIVEVLLKAGADVNAQDKFGKTAFDISIDNGNEDLAEILQKLN

SEQ ID NO: 116 - protein, 103 aa, synthetic construct
MRGSHHHHHHDADLAKKLLAAAAAGQLELVEQLLKAGADVNAKDKDGYTPLHLAAREGHLEIVEVLLKAGADVNAQDKFGKTAFDISIDNGNEDLAEILQKLN

SEQ ID NO: 117 - protein, 169 aa, synthetic construct
MRGSHHHHHHGSDLGKKLLQAARAGQLDEVRELLKAGADVNAKDREGITPLHLAAQHGHLEIVEVLLKAGADVNAKDVWGRTPLHLAAWQGHLEIVEVLLKAGADVNAKDLAGATPLHVAALYGHLEIVEVLLKAGADVNAQDKSGKTPADLAARAGHQDIAEVLQKAA

SEQ ID NO: 118 - protein, 169 aa, synthetic construct
MRGSHHHHHHGSDLGKKLLQAARAGQLDLVRELLKAGADVNAKDREGITPLHLAAQHGHLEIVEVLLKAGADVNAKDVWGRTPLHLAAWQGHLEIVEVLLKAGADVNAKDLAGATPLHVAALYGHLEIVEVLLKAGADVNAQDKSGKTPADLAARAGHQDIAEVLQKAA

SEQ ID NO: 119 - protein, 136 aa, synthetic construct
MRGSHHHHHHGSDLGKKLLEAARAGQIDEVKELIALGADVNAKDYFSHTPLHLAARNGHIKIVKLLLEAGADVNAKDFAGKTPLHLAANEGHIEIVKLLLEAGADVNAQDIFGKTPADIAAAAGHEDIVKLLQEAA

SEQ ID NO: 120 - protein, 136 aa, synthetic construct
MRGSHHHHHHGSDLGKKLLEAARAGQIDLVKELIALGADVNAKDYFSHTPLHLAARNGHIKIVKLLLEAGADVNAKDFAGKTPLHLAANEGHIEIVKLLLEAGADVNAQDIFGKTPADIAAAAGHEDIVKLLQEAA

SEQ ID NO: 121 - protein, 136 aa, synthetic construct
MRGSHHHHHHGSDLGKKLLEAARAGQIDEVKKLIALGADVNAKDYFSHTPLHLAARNGHIKIVKLLLEAGADVNAKDFAGKTPLHLAANEGHIEIVKLLLEAGADVNAQDIFGKTPADIAAAAGHEDIVKLLQEAA

SEQ ID NO: 122 - protein, 136 aa, synthetic construct
MRGSHHHHHHGSDLGKKLLEAARAGQIDLVKKLIALGADVNAKDYFSHTPLHLAARNGHIKIVKLLLEAGADVNAKDFAGKTPLHLAANEGHIEIVKLLLEAGADVNAQDIFGKTPADIAAAAGHEDIVKLLQEAA

SEQ ID NO: 123 - protein, 136 aa, synthetic construct
MRGSHHHHHHGSDLGKKLLEAAAAGQIDEVKELIALGADVNAKDLYGSTPLHLAALRGHIEIVKLLLEAGADVNAKDVWGLTPLHLAALEGHLEIVKLLLEAGADVNAQDSAGKTPADLAALAGHEEIAELLQKAA

SEQ ID NO: 124 - protein, 136 aa, synthetic construct
MRGSHHHHHHGSDLGKKLLEAAAAGQIDLVKELIALGADVNAKDLYGSTPLHLAALRGHIEIVKLLLEAGADVNAKDVWGLTPLHLAALEGHLEIVKLLLEAGADVNAQDSAGKTPADLAALAGHEEIAELLQKAA

SEQ ID NO: 125 - protein, 136 aa, synthetic construct
MRGSHHHHHHGSDLGKKLLEAAAAGQIDEVKELIALGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAA

SEQ ID NO: 126 - protein, 136 aa, synthetic construct
MRGSHHHHHHGSDLGKKLLEAAAAGQIDLVKELIALGADVNAKDYFSHTPLHLAARNGHLKIVEVLLKAGADVNAKDFAGKTPLHLAANEGHLEIVEVLLKAGADVNAQDIFGKTPADIAADAGHEDIAEVLQKAA

SEQ ID NO: 127 - protein, 136 aa, synthetic construct
MRGSHHHHHHGSDLDKKLLEAARAGQDDEVRILLKAGADVNAKDSTGWTPLHLAAPWGHPEIVEVLLKAGADVNAKDFQGWTPLHLAAAAGHLEIVEVLLKAGADVNAQDKSGKTPADLAADAGHEDIAEVLQKAA

SEQ ID NO: 128 - protein, 136 aa, synthetic construct
MRGSHHHHHHGSDLDKKLLEAARAGQDDLVRILLKAGADVNAKDSTGWTPLHLAAPWGHPEIVEVLLKAGADVNAKDFQGWTPLHLAAAAGHLEIVEVLLKAGADVNAQDKSGKTPADLAADAGHEDIAEVLQKAA

SEQ ID NO: 129 - protein, 136 aa, synthetic construct
MRGSHHHHHHGSDLDKKLLEAAAAGQIDEVKELIALGADVNAKDSTGWTPLHLAAPWGHPEIVKLLLEAGADVNAKDFQGWTPLHLAAAAGHLEIVKLLLEAGADVNAQDKSGKTPADLAADAGHEDIVKLLQEAA

SEQ ID NO: 130 - protein, 136 aa, synthetic construct
MRGSHHHHHHGSDLDKKLLEAAAAGQIDLVKELIALGADVNAKDSTGWTPLHLAAPWGHPEIVKLLLEAGADVNAKDFQGWTPLHLAAAAGHLEIVKLLLEAGADVNAQDKSGKTPADLAADAGHEDIVKLLQEAA

SEQ ID NO: 131 - protein, 169 aa, synthetic construct
MRGSHHHHHHGSDLGKKLLQAAAAGQIDEVKELIALGADVNAKDREGITPLHLAAQHGHIEIVKLLLEAGADVNAKDVWGRTPLHLAAWQGHIEIVKLLLEAGADVNAKDLAGATPLHVAALYGHLEIVKLLLEAGADVNAQDKSGKTPADLAARAGHQDIVKLLQEAA

SEQ ID NO: 132 - protein, 169 aa, synthetic construct
MRGSHHHHHHGSDLGKKLLQAAAAGQIDLVKELIALGADVNAKDREGITPLHLAAQHGHIEIVKLLLEAGADVNAKDVWGRTPLHLAAWQGHIEIVKLLLEAGADVNAKDLAGATPLHVAALYGHLEIVKLLLEAGADVNAQDKSGKTPADLAARAGHQDIVKLLQEAA